SEQUENCE LISTING (14 sequences)

SEQ ID NO: 1 - DNA, 3687 bp, Bacillus thuringiensis
  CDS (1)..(3687)
ttgacttcaaataggaaaaatgagaatgaaattataaatgctttatcgattccaacggtatcgaatccttccacgcaaatgaatctatcaccagatgctcgtattgaagatagcttgtgtgtagccgaggtgaacaatattgatccatttgttagcgcatcaacagtccaaacgggtataaacatagctggtagaatattgggcgtattaggtgtgccgtttgctggacaactagctagtttttatagttttcttgttggggaattatggcctagtggcagagatccatgggaaattttcctggaacatgtagaacaacttataagacaacaagtaacagaaaatactaggaatacggctattgctcgattagaaggtctaggaagaggctatagatcttaccagcaggctcttgaaacttggttagataaccgaaatgatgcaagatcaagaagcattattcttgagcgctatgttgctttagaacttgacattactactgctataccgcttttcagaatacgaaatgaagaagttccattattaatggtatatgctcaagctgcaaatttacacctattattattgagagacgcatccctttttggtagtgaatgggggatggcatcttccgatgttaaccaatattaccaagaacaaatcagatatacagaggaatattctaaccattgcgtacaatggtataatacagggctaaataacttaagagggacaaatgctgaaagttggttgcggtataatcaattccgtagagacctaacgttaggggtattagatttagtagccctattcccaagctatgatactcgcacttatccaatcaatacgagtgctcagttaacaagagaaatttatacagatccaattgggagaacaaatgcaccttcaggatttgcaagtacgaattggtttaataataatgcaccatcgttttctgccatagaggctgccattttcaggcctccgcatctacttgattttccagaacaacttacaatttacagtgcatcaagccgttggagtagcactcaacatatgaattattgggtgggacataggcttaacttccgcccaataggagggacattaaatacctcaacacaaggacttactaataatacttcaattaatcctgtaacattacagtttacgtctcgagacgtttatagaacagaatcaaatgcagggacaaatatactatttactactcctgtgaatggagtaccttgggctagatttaattttataaaccctcagaatatttatgaaagaggcgccactacctacagtcaaccgtatcagggagttgggattcaattatttgattcagaaactgaattaccaccagaaacaacagaacgaccaaattatgaatcatatagtcatagattatctcatataggactaatcataggaaacactttgagagcaccagtctattcttggacgcatcgtagtgcagatcgtacgaatacgattggaccaaatagaattacacaaataccattggtaaaagcactgaatcttcattcaggtgttactgttgttggagggccaggatttacaggtggggatatccttcgtagaacaaatacgggtacatttggagatatacgattaaatattaatgtgccattatcccaaagatatcgcgtaaggattcgttatgcttctactacagatttacaatttttcacgagaattaatggaaccactgttaatattggtaatttctcaagaactatgaataggggggataatttagaatatagaagttttagaactgcaggatttagtactccttttaattttttaaatgcccaaagcacattcacattgggtgctcagagtttttcaaatcaggaagtttatatagatagagtcgaatttgttccagcagaggtaacatttgaggcagaatatgatttagaaagagcacaaaaggcggtgaatgctctgtttacttctacaaatccaagaagattgaaaacagatgtgacagattatcatattgaccaagtgtccaatatggtggcatgtttatcagatgaattttgcttggatgagaagcgagaattatttgagaaagtgaaatatgcgaagcgactcagtgatgaaagaaacttactccaagatccaaacttcacattcatcagtgggcaattaagtttcgcatccatcgatggacaatcaaacttcccctctattaatgagctatctgaacatggatggtggggaagtgcgaatgttaccattcaggaagggaatgacgtatttaaagagaattacgtcacactaccgggtacttttaatgagtgttatccaaattatttatatcaaaaaataggagagtcagaattaaaagcttatacgcgctatcaattaagagggtatattgaagatagtcaagatctagagatttatttaattcgttacaatgcaaagcatgaaacattggatgttccaggtaccgattccctatggccgctttcagttgaaagcccaatcggaaggtgcggagaaccaaatcgatgcgcaccacattttgaatggaatcctgatctagattgttcctgcagagatggagaaagatgtgcgcatcattcccatcatttcactttggatattgatgttgggtgcacagacttgcatgagaacctaggcgtgtgggtggtattcaagattaagacgcaggaaggttatgcaagattaggaaatctggaatttatcgaagagaaaccattaattggagaagcactgtctcgtgtgaagagagcggaaaaaaaatggagagacaaacgggaaaaactacaattggaaacaaaacgagtatatacagaggcaaaagaagctgtggatgctttattcgtagattctcaatatgatcaattacaagcggatacaaacattggcatgattcatgcggcagataaacttgttcatcgaattcgagaggcgtatctttcagaattacctgttatcccaggtgtaaatgcggaaatttttgaagaattagaaggtcacattatcactgcaatgtccttatacgatgcgagaaatgtcgttaaaaatggtgattttaataatggattaacatgttggaatgtaaaagggcatgtagatgtacaacagagccatcatcgttctgaccttgttatcccagaatgggaagcagaagtgtcacaagcagttcgcgtctgtccggggcgtggctatatccttcgtgtcacagcgtacaaagagggatatggagagggctgcgtaacgatccatgaaatcgagaacaatacagacgaactaaaatttaaaaactgtgaagaagaggaagtgtatccaacggatacaggaacgtgtaatgattatactgcacaccaaggtacagcagcatgtaattcccgtaatgctggatatgaggatgcatatgaagttgatactacagcatctgttaattacaaaccgacttatgaagaagaaacgtatacagatgtacgaagagataatcattgtgaatatgacagagggtatgtgaattatccaccagtaccagctggttatgtgacaaaagaattagaatacttcccagaaacagatacagtatggattgagattggagaaacggaaggaaagtttattgtagatagcgtggaactactcctcatggaagaa

SEQ ID NO: 2 - protein, 1229 aa, Bacillus thuringiensis
  misc_feature (1)..(864): sequence encoding toxin domain I
LTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSDEFCLDEKRELFEKVKYAKRLSDERNLLQDPNFTFISGQLSFASIDGQSNFPSINELSEHGWWGSANVTIQEGNDVFKENYVTLPGTFNECYPNYLYQKIGESELKAYTRYQLRGYIEDSQDLEIYLIRYNAKHETLDVPGTDSLWPLSVESPIGRCGEPNRCAPHFEWNPDLDCSCRDGERCAHHSHHFTLDIDVGCTDLHENLGVWVVFKIKTQEGYARLGNLEFIEEKPLIGEALSRVKRAEKKWRDKREKLQLETKRVYTEAKEAVDALFVDSQYDQLQADTNIGMIHAADKLVHRIREAYLSELPVIPGVNAEIFEELEGHIITAMSLYDARNVVKNGDFNNGLTCWNVKGHVDVQQSHHRSDLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 3 - DNA, 3690 bp, artificial sequence
  fully synthetic coding sequence
atggccacctccaaccgcaagaacgagaatgagatcatcaacgccctgtcgatccccacggtctcgaacccgtccacccaaatgaacctgtccccggacgcccgcatcgaggactccctgtgcgtcgcggaggtcaacaacatcgaccccttcgtctccgcctccacggtccagacgggcatcaacatcgctggccgcatcctcggcgtcctgggcgtcccgttcgctggccagctggcctccttctactccttcctggtcggggagctgtggccctccggtcgcgacccctgggagatcttcctggagcacgtcgagcagctcatccgccagcaagtcaccgagaacacccgcaacacggccatcgcccgcctggagggcctgggccgtggctaccgctcctaccagcaggccctggagacctggctggacaaccgcaacgacgcacgctcccgctccatcatcctggagcgctacgtggcgctggagctggacatcaccaccgccatcccgctcttccgcatccgcaatgaagaggtgcccctgctcatggtctacgcccaggctgccaacctgcacctgctcctgcttcgcgatgcatccctgttcggctccgagtggggcatggcctcgtccgacgtcaaccagtactatcaggagcagatccgctacaccgaggagtactccaaccactgcgtccagtggtacaacaccggcctcaacaacctgcgcggcacgaacgctgagtcctggctgcgctacaaccagttccgccgcgacctgacgctgggcgtcctggacctggtcgccctcttcccctcctacgacacccgcacctaccccatcaacacgtccgcccagctgacccgcgagatctacaccgaccccatcggccgcaccaacgctccctccggcttcgcgtccacgaactggttcaacaacaatgccccgtcgttctccgccatcgaggctgcgatcttccgcccaccgcacctcctggacttccccgagcagctgaccatctactccgcctcgtcccgctggtcgtccacccagcacatgaactactgggtgggccaccgcctcaacttcaggcccatcggtggcaccctgaacacctccacccagggcctgaccaacaacacctccatcaaccccgtcaccctccagttcacgtcccgcgacgtctaccgcaccgagtccaacgccggcaccaacatcctcttcacgaccccggtcaacggcgtcccctgggctcgcttcaacttcatcaacccgcagaacatctacgagcgtggtgcgaccacctactcccagccgtaccagggcgtcggcatccagctcttcgactccgagaccgagctgccacccgagacgaccgagcgtcccaactacgagtcctactcccaccgcctgtcccacatcggcctgatcatcggcaacaccctcagggctcccgtctactcctggacgcaccgctccgcggaccgcacgaacacgatcggtcccaaccgcatcacccagatccccctggtcaaggccctcaacctgcactccggcgtcaccgtcgtgggtggcccaggcttcaccggtggcgacatcctgcgcaggaccaacacgggcaccttcggcgacatccgcctcaacatcaacgtcccgctgtcccagcgctaccgcgtccgcatccgctacgcctccacgaccgacctccagttcttcacgcgcatcaacggcaccacggtcaacatcggcaacttctcccgcaccatgaacaggggcgacaacctggagtaccgctccttccgcaccgccggcttctccaccccgttcaacttcctcaacgcccagtccaccttcacccttggtgcgcagtccttctccaaccaggaggtctacatcgaccgcgtcgagttcgtcccagccgaggtcaccttcgaggccgagtacgacctggagcgtgcccagaaggcggtgaacgccctgttcacctccaccaaccccaggcgcctgaagaccgacgtcacggactaccacatcgaccaggtgtccaacatggtggcctgcctctccgacgagttctgcctggacgagaagcgcgagctgttcgagaaggtcaagtacgcgaagcgcctctccgacgagcgcaacctgctccaggacccgaacttcaccttcatctccggccagctgtccttcgcgtccatcgacggccagtccaacttcccctccatcaacgagctgtccgagcacggctggtggggctccgcgaacgtcaccatccaggagggcaacgacgtcttcaaggagaactacgtcaccctgccgggcaccttcaacgagtgctacccgaactacctctaccagaagatcggcgagtccgagctgaaggcctacacccgctaccagctgcgcggctacatcgaggactcccaggacctggagatctacctcatccgctacaacgcgaagcacgagaccctggacgtccctggcacggactccctgtggcccctctccgtcgagtcgcccatcggccgctgcggcgagcccaaccgctgcgctccccacttcgagtggaaccccgacctggactgctcctgccgcgacggcgagcgctgcgcgcaccattcccatcacttcaccctggacatcgacgtcggctgcaccgacctgcacgagaacctgggcgtgtgggtggtcttcaagatcaagacgcaggagggctacgcccgcctgggcaacctggagttcatcgaggagaagccgctgatcggcgaggcgctctcccgcgtcaagcgtgcggagaagaagtggcgcgacaagcgcgagaagctccagctggagaccaagcgcgtctacaccgaggccaaggaggccgtggacgccctgttcgtcgactcccagtacgaccagctccaggcggacaccaacatcggcatgatccatgcggctgacaagctggtccaccgcatccgcgaggcgtacctgtccgagctgcccgtcatccctggcgtcaacgcggagatcttcgaggagctggagggccacatcatcaccgccatgtccctctacgacgcgcgcaacgtggtcaagaacggcgacttcaacaacggcctgacgtgctggaacgtcaagggccacgtcgacgtccagcaatcccaccaccgctccgacctggtcatccccgagtgggaggccgaggtgtcccaggccgtccgcgtctgtccgggcaggggctacatcctgcgcgtcaccgcgtacaaggagggctacggcgagggctgcgtcacgatccacgagatcgagaacaacaccgacgagctgaagttcaagaactgcgaggaggaggaggtctacccgacggacaccggcacgtgcaacgactacaccgcgcaccagggcaccgctgcctgcaactcccgcaacgctggctacgaggacgcctacgaggtcgacaccaccgcctccgtcaactacaagccgacctacgaggaggagacctacaccgacgtccgtcgcgacaaccactgcgagtacgaccgcggctacgtgaactacccacccgtccccgctggctacgtcacgaaggagctggagtacttccccgagaccgacaccgtctggatcgagatcggcgagacggagggcaagttcatcgtcgactccgtcgagctgctcctgatggaggag

SEQ ID NO: 4 - protein, 1230 aa, artificial sequence
  fully synthetic coding sequence
MATSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSDEFCLDEKRELFEKVKYAKRLSDERNLLQDPNFTFISGQLSFASIDGQSNFPSINELSEHGWWGSANVTIQEGNDVFKENYVTLPGTFNECYPNYLYQKIGESELKAYTRYQLRGYIEDSQDLEIYLIRYNAKHETLDVPGTDSLWPLSVESPIGRCGEPNRCAPHFEWNPDLDCSCRDGERCAHHSHHFTLDIDVGCTDLHENLGVWVVFKIKTQEGYARLGNLEFIEEKPLIGEALSRVKRAEKKWRDKREKLQLETKRVYTEAKEAVDALFVDSQYDQLQADTNIGMIHAADKLVHRIREAYLSELPVIPGVNAEIFEELEGHIITAMSLYDARNVVKNGDFNNGLTCWNVKGHVDVQQSHHRSDLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 5 - DNA, 6600 bp, Artificial Sequence
  fully synthetic expression cassette
gcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgcggccacgcgtggtaccaagcttcccgatcctatctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggaatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccggatccccatgggaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatatccccgcggccgcgttaacaagcttgagctcaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactgaaccaatccttctagaagatcgtgtccacccacccctcgatctctcgctcgccgccgccgatcggatcgcgtggttggatcatcacaactcggcaaagagatctgagctcatcaggtgaggattaggattccaaataagcgataacgtttacctggtcactgcgattagttcagtttactgtgaaattctttggacccttcttaattataaatttgcttgttttctcggcagattcctcaatgccggtctagaggatctccatggccacctccaaccgcaagaacgagaatgagatcatcaacgccctgtcgatccccacggtctcgaacccgtccacccaaatgaacctgtccccggacgcccgcatcgaggactccctgtgcgtcgcggaggtcaacaacatcgaccccttcgtctccgcctccacggtccagacgggcatcaacatcgctggccgcatcctcggcgtcctgggcgtcccgttcgctggccagctggcctccttctactccttcctggtcggggagctgtggccctccggtcgcgacccctgggagatcttcctggagcacgtcgagcagctcatccgccagcaagtcaccgagaacacccgcaacacggccatcgcccgcctggagggcctgggccgtggctaccgctcctaccagcaggccctggagacctggctggacaaccgcaacgacgcacgctcccgctccatcatcctggagcgctacgtggcgctggagctggacatcaccaccgccatcccgctcttccgcatccgcaatgaagaggtgcccctgctcatggtctacgcccaggctgccaacctgcacctgctcctgcttcgcgatgcatccctgttcggctccgagtggggcatggcctcgtccgacgtcaaccagtactatcaggagcagatccgctacaccgaggagtactccaaccactgcgtccagtggtacaacaccggcctcaacaacctgcgcggcacgaacgctgagtcctggctgcgctacaaccagttccgccgcgacctgacgctgggcgtcctggacctggtcgccctcttcccctcctacgacacccgcacctaccccatcaacacgtccgcccagctgacccgcgagatctacaccgaccccatcggccgcaccaacgctccctccggcttcgcgtccacgaactggttcaacaacaatgccccgtcgttctccgccatcgaggctgcgatcttccgcccaccgcacctcctggacttccccgagcagctgaccatctactccgcctcgtcccgctggtcgtccacccagcacatgaactactgggtgggccaccgcctcaacttcaggcccatcggtggcaccctgaacacctccacccagggcctgaccaacaacacctccatcaaccccgtcaccctccagttcacgtcccgcgacgtctaccgcaccgagtccaacgccggcaccaacatcctcttcacgaccccggtcaacggcgtcccctgggctcgcttcaacttcatcaacccgcagaacatctacgagcgtggtgcgaccacctactcccagccgtaccagggcgtcggcatccagctcttcgactccgagaccgagctgccacccgagacgaccgagcgtcccaactacgagtcctactcccaccgcctgtcccacatcggcctgatcatcggcaacaccctcagggctcccgtctactcctggacgcaccgctccgcggaccgcacgaacacgatcggtcccaaccgcatcacccagatccccctggtcaaggccctcaacctgcactccggcgtcaccgtcgtgggtggcccaggcttcaccggtggcgacatcctgcgcaggaccaacacgggcaccttcggcgacatccgcctcaacatcaacgtcccgctgtcccagcgctaccgcgtccgcatccgctacgcctccacgaccgacctccagttcttcacgcgcatcaacggcaccacggtcaacatcggcaacttctcccgcaccatgaacaggggcgacaacctggagtaccgctccttccgcaccgccggcttctccaccccgttcaacttcctcaacgcccagtccaccttcacccttggtgcgcagtccttctccaaccaggaggtctacatcgaccgcgtcgagttcgtcccagccgaggtcaccttcgaggccgagtacgacctggagcgtgcccagaaggcggtgaacgccctgttcacctccaccaaccccaggcgcctgaagaccgacgtcacggactaccacatcgaccaggtgtccaacatggtggcctgcctctccgacgagttctgcctggacgagaagcgcgagctgttcgagaaggtcaagtacgcgaagcgcctctccgacgagcgcaacctgctccaggacccgaacttcaccttcatctccggccagctgtccttcgcgtccatcgacggccagtccaacttcccctccatcaacgagctgtccgagcacggctggtggggctccgcgaacgtcaccatccaggagggcaacgacgtcttcaaggagaactacgtcaccctgccgggcaccttcaacgagtgctacccgaactacctctaccagaagatcggcgagtccgagctgaaggcctacacccgctaccagctgcgcggctacatcgaggactcccaggacctggagatctacctcatccgctacaacgcgaagcacgagaccctggacgtccctggcacggactccctgtggcccctctccgtcgagtcgcccatcggccgctgcggcgagcccaaccgctgcgctccccacttcgagtggaaccccgacctggactgctcctgccgcgacggcgagcgctgcgcgcaccattcccatcacttcaccctggacatcgacgtcggctgcaccgacctgcacgagaacctgggcgtgtgggtggtcttcaagatcaagacgcaggagggctacgcccgcctgggcaacctggagttcatcgaggagaagccgctgatcggcgaggcgctctcccgcgtcaagcgtgcggagaagaagtggcgcgacaagcgcgagaagctccagctggagaccaagcgcgtctacaccgaggccaaggaggccgtggacgccctgttcgtcgactcccagtacgaccagctccaggcggacaccaacatcggcatgatccatgcggctgacaagctggtccaccgcatccgcgaggcgtacctgtccgagctgcccgtcatccctggcgtcaacgcggagatcttcgaggagctggagggccacatcatcaccgccatgtccctctacgacgcgcgcaacgtggtcaagaacggcgacttcaacaacggcctgacgtgctggaacgtcaagggccacgtcgacgtccagcaatcccaccaccgctccgacctggtcatccccgagtgggaggccgaggtgtcccaggccgtccgcgtctgtccgggcaggggctacatcctgcgcgtcaccgcgtacaaggagggctacggcgagggctgcgtcacgatccacgagatcgagaacaacaccgacgagctgaagttcaagaactgcgaggaggaggaggtctacccgacggacaccggcacgtgcaacgactacaccgcgcaccagggcaccgctgcctgcaactcccgcaacgctggctacgaggacgcctacgaggtcgacaccaccgcctccgtcaactacaagccgacctacgaggaggagacctacaccgacgtccgtcgcgacaaccactgcgagtacgaccgcggctacgtgaactacccacccgtccccgctggctacgtcacgaaggagctggagtacttccccgagaccgacaccgtctggatcgagatcggcgagacggagggcaagttcatcgtcgactccgtcgagctgctcctgatggaggagtgatagaattctaaatcttattattatcatcgtcgtcgtcgtctcgtcacggaattaattaaagtacctactccgtacttagctagctacaataataaggattcattgatcactacaagagtgatcgactcgactgtagtatgtgtgtgcaatataatgtgctgtctatcaacaactactagtattgtcatttttttcgaaccagggaactttttaatgataagaagaaaaagacaagtacttattgtcgagcatgcgtgtgtgttt

SEQ ID NO: 6 - protein, 264 aa, Artificial Sequence [sequence omitted]

SEQ ID NO: 7 - protein, 1230 aa, Artificial Sequence; identical to SEQ ID NO: 4
  fully synthetic expression cassette

SEQ ID NO: 8 - DNA, 7000 bp, Artificial Sequence
  fully synthetic expression cassette
gcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgcggccacgcgtggtaccaagcttcccgatcctatctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggaatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccggatccccatgggaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatatccccgcggccgcgttaacaagcttgagctcaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactgaaccaatccttctagaagatcgtgtccacccacccctcgatctctcgctcgccgccgccgatcggatcgcgtggttggatcatcacaactcggcaaagagatctgagctcatcaggtgaggattaggattccaaataagcgataacgtttacctggtcactgcgattagttcagtttactgtgaaattctttggacccttcttaattataaatttgcttgttttctcggcagattcctcaatgccggtctagaggatcagcatggcgcccaccgtgatgatggcctcgtcggccaccgccgtcgctccgttcctggggctcaagtccaccgccagcctccccgtcgcccgccgctcctccagaagcctcggcaacgtcagcaacggcggaaggatccggtgcatgcaggtaacaaatgcatcctagctagtagttctttgcattgcagcagctgcagctagcgagttagtaataggaagggaactgatgatccatgcatggactgatgtgtgttgcccatcccatcccatcccatttcccaaacgaaccgaaaacaccgtactacgtgcaggtgtggccctacggcaacaagaagttcgagacgctgtcgtacctgccgccgctgtcgaccggcgggcgcatccgctgcatgcaggccatggccacctccaaccgcaagaacgagaatgagatcatcaacgccctgtcgatccccacggtctcgaacccgtccacccaaatgaacctgtccccggacgcccgcatcgaggactccctgtgcgtcgcggaggtcaacaacatcgaccccttcgtctccgcctccacggtccagacgggcatcaacatcgctggccgcatcctcggcgtcctgggcgtcccgttcgctggccagctggcctccttctactccttcctggtcggggagctgtggccctccggtcgcgacccctgggagatcttcctggagcacgtcgagcagctcatccgccagcaagtcaccgagaacacccgcaacacggccatcgcccgcctggagggcctgggccgtggctaccgctcctaccagcaggccctggagacctggctggacaaccgcaacgacgcacgctcccgctccatcatcctggagcgctacgtggcgctggagctggacatcaccaccgccatcccgctcttccgcatccgcaatgaagaggtgcccctgctcatggtctacgcccaggctgccaacctgcacctgctcctgcttcgcgatgcatccctgttcggctccgagtggggcatggcctcgtccgacgtcaaccagtactatcaggagcagatccgctacaccgaggagtactccaaccactgcgtccagtggtacaacaccggcctcaacaacctgcgcggcacgaacgctgagtcctggctgcgctacaaccagttccgccgcgacctgacgctgggcgtcctggacctggtcgccctcttcccctcctacgacacccgcacctaccccatcaacacgtccgcccagctgacccgcgagatctacaccgaccccatcggccgcaccaacgctccctccggcttcgcgtccacgaactggttcaacaacaatgccccgtcgttctccgccatcgaggctgcgatcttccgcccaccgcacctcctggacttccccgagcagctgaccatctactccgcctcgtcccgctggtcgtccacccagcacatgaactactgggtgggccaccgcctcaacttcaggcccatcggtggcaccctgaacacctccacccagggcctgaccaacaacacctccatcaaccccgtcaccctccagttcacgtcccgcgacgtctaccgcaccgagtccaacgccggcaccaacatcctcttcacgaccccggtcaacggcgtcccctgggctcgcttcaacttcatcaacccgcagaacatctacgagcgtggtgcgaccacctactcccagccgtaccagggcgtcggcatccagctcttcgactccgagaccgagctgccacccgagacgaccgagcgtcccaactacgagtcctactcccaccgcctgtcccacatcggcctgatcatcggcaacaccctcagggctcccgtctactcctggacgcaccgctccgcggaccgcacgaacacgatcggtcccaaccgcatcacccagatccccctggtcaaggccctcaacctgcactccggcgtcaccgtcgtgggtggcccaggcttcaccggtggcgacatcctgcgcaggaccaacacgggcaccttcggcgacatccgcctcaacatcaacgtcccgctgtcccagcgctaccgcgtccgcatccgctacgcctccacgaccgacctccagttcttcacgcgcatcaacggcaccacggtcaacatcggcaacttctcccgcaccatgaacaggggcgacaacctggagtaccgctccttccgcaccgccggcttctccaccccgttcaacttcctcaacgcccagtccaccttcacccttggtgcgcagtccttctccaaccaggaggtctacatcgaccgcgtcgagttcgtcccagccgaggtcaccttcgaggccgagtacgacctggagcgtgcccagaaggcggtgaacgccctgttcacctccaccaaccccaggcgcctgaagaccgacgtcacggactaccacatcgaccaggtgtccaacatggtggcctgcctctccgacgagttctgcctggacgagaagcgcgagctgttcgagaaggtcaagtacgcgaagcgcctctccgacgagcgcaacctgctccaggacccgaacttcaccttcatctccggccagctgtccttcgcgtccatcgacggccagtccaacttcccctccatcaacgagctgtccgagcacggctggtggggctccgcgaacgtcaccatccaggagggcaacgacgtcttcaaggagaactacgtcaccctgccgggcaccttcaacgagtgctacccgaactacctctaccagaagatcggcgagtccgagctgaaggcctacacccgctaccagctgcgcggctacatcgaggactcccaggacctggagatctacctcatccgctacaacgcgaagcacgagaccctggacgtccctggcacggactccctgtggcccctctccgtcgagtcgcccatcggccgctgcggcgagcccaaccgctgcgctccccacttcgagtggaaccccgacctggactgctcctgccgcgacggcgagcgctgcgcgcaccattcccatcacttcaccctggacatcgacgtcggctgcaccgacctgcacgagaacctgggcgtgtgggtggtcttcaagatcaagacgcaggagggctacgcccgcctgggcaacctggagttcatcgaggagaagccgctgatcggcgaggcgctctcccgcgtcaagcgtgcggagaagaagtggcgcgacaagcgcgagaagctccagctggagaccaagcgcgtctacaccgaggccaaggaggccgtggacgccctgttcgtcgactcccagtacgaccagctccaggcggacaccaacatcggcatgatccatgcggctgacaagctggtccaccgcatccgcgaggcgtacctgtccgagctgcccgtcatccctggcgtcaacgcggagatcttcgaggagctggagggccacatcatcaccgccatgtccctctacgacgcgcgcaacgtggtcaagaacggcgacttcaacaacggcctgacgtgctggaacgtcaagggccacgtcgacgtccagcaatcccaccaccgctccgacctggtcatccccgagtgggaggccgaggtgtcccaggccgtccgcgtctgtccgggcaggggctacatcctgcgcgtcaccgcgtacaaggagggctacggcgagggctgcgtcacgatccacgagatcgagaacaacaccgacgagctgaagttcaagaactgcgaggaggaggaggtctacccgacggacaccggcacgtgcaacgactacaccgcgcaccagggcaccgctgcctgcaactcccgcaacgctggctacgaggacgcctacgaggtcgacaccaccgcctccgtcaactacaagccgacctacgaggaggagacctacaccgacgtccgtcgcgacaaccactgcgagtacgaccgcggctacgtgaactacccacccgtccccgctggctacgtcacgaaggagctggagtacttccccgagaccgacaccgtctggatcgagatcggcgagacggagggcaagttcatcgtcgactccgtcgagctgctcctgatggaggagtgatagaattctaaatcttattattatcatcgtcgtcgtcgtctcgtcacggaattaattaaagtacctactccgtacttagctagctacaataataaggattcattgatcactacaagagtgatcgactcgactgtagtatgtgtgtgcaatataatgtgctgtctatcaacaactactagtattgtcatttttttcgaaccagggaactttttaatgataagaagaaaaagacaagtacttattgtcgagcatgcgtgtgtgtttttt

SEQ ID NO: 9 - protein, 264 aa, Artificial Sequence [sequence omitted]

SEQ ID NO: 10 - protein, 1230 aa, Artificial Sequence; identical to SEQ ID NO: 4
  fully synthetic expression cassette

SEQ ID NO: 11 - DNA, 5170 bp, Artificial Sequence
  fully synthetic expression cassette
gcggccgcgttaacaagcttctgcaggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatcctctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggccacctccaaccgcaagaacgagaatgagatcatcaacgccctgtcgatccccacggtctcgaacccgtccacccaaatgaacctgtccccggacgcccgcatcgaggactccctgtgcgtcgcggaggtcaacaacatcgaccccttcgtctccgcctccacggtccagacgggcatcaacatcgctggccgcatcctcggcgtcctgggcgtcccgttcgctggccagctggcctccttctactccttcctggtcggggagctgtggccctccggtcgcgacccctgggagatcttcctggagcacgtcgagcagctcatccgccagcaagtcaccgagaacacccgcaacacggccatcgcccgcctggagggcctgggccgtggctaccgctcctaccagcaggccctggagacctggctggacaaccgcaacgacgcacgctcccgctccatcatcctggagcgctacgtggcgctggagctggacatcaccaccgccatcccgctcttccgcatccgcaatgaagaggtgcccctgctcatggtctacgcccaggctgccaacctgcacctgctcctgcttcgcgatgcatccctgttcggctccgagtggggcatggcctcgtccgacgtcaaccagtactatcaggagcagatccgctacaccgaggagtactccaaccactgcgtccagtggtacaacaccggcctcaacaacctgcgcggcacgaacgctgagtcctggctgcgctacaaccagttccgccgcgacctgacgctgggcgtcctggacctggtcgccctcttcccctcctacgacacccgcacctaccccatcaacacgtccgcccagctgacccgcgagatctacaccgaccccatcggccgcaccaacgctccctccggcttcgcgtccacgaactggttcaacaacaatgccccgtcgttctccgccatcgaggctgcgatcttccgcccaccgcacctcctggacttccccgagcagctgaccatctactccgcctcgtcccgctggtcgtccacccagcacatgaactactgggtgggccaccgcctcaacttcaggcccatcggtggcaccctgaacacctccacccagggcctgaccaacaacacctccatcaaccccgtcaccctccagttcacgtcccgcgacgtctaccgcaccgagtccaacgccggcaccaacatcctcttcacgaccccggtcaacggcgtcccctgggctcgcttcaacttcatcaacccgcagaacatctacgagcgtggtgcgaccacctactcccagccgtaccagggcgtcggcatccagctcttcgactccgagaccgagctgccacccgagacgaccgagcgtcccaactacgagtcctactcccaccgcctgtcccacatcggcctgatcatcggcaacaccctcagggctcccgtctactcctggacgcaccgctccgcggaccgcacgaacacgatcggtcccaaccgcatcacccagatccccctggtcaaggccctcaacctgcactccggcgtcaccgtcgtgggtggcccaggcttcaccggtggcgacatcctgcgcaggaccaacacgggcaccttcggcgacatccgcctcaacatcaacgtcccgctgtcccagcgctaccgcgtccgcatccgctacgcctccacgaccgacctccagttcttcacgcgcatcaacggcaccacggtcaacatcggcaacttctcccgcaccatgaacaggggcgacaacctggagtaccgctccttccgcaccgccggcttctccaccccgttcaacttcctcaacgcccagtccaccttcacccttggtgcgcagtccttctccaaccaggaggtctacatcgaccgcgtcgagttcgtcccagccgaggtcaccttcgaggccgagtacgacctggagcgtgcccagaaggcggtgaacgccctgttcacctccaccaaccccaggcgcctgaagaccgacgtcacggactaccacatcgaccaggtgtccaacatggtggcctgcctctccgacgagttctgcctggacgagaagcgcgagctgttcgagaaggtcaagtacgcgaagcgcctctccgacgagcgcaacctgctccaggacccgaacttcaccttcatctccggccagctgtccttcgcgtccatcgacggccagtccaacttcccctccatcaacgagctgtccgagcacggctggtggggctccgcgaacgtcaccatccaggagggcaacgacgtcttcaaggagaactacgtcaccctgccgggcaccttcaacgagtgctacccgaactacctctaccagaagatcggcgagtccgagctgaaggcctacacccgctaccagctgcgcggctacatcgaggactcccaggacctggagatctacctcatccgctacaacgcgaagcacgagaccctggacgtccctggcacggactccctgtggcccctctccgtcgagtcgcccatcggccgctgcggcgagcccaaccgctgcgctccccacttcgagtggaaccccgacctggactgctcctgccgcgacggcgagcgctgcgcgcaccattcccatcacttcaccctggacatcgacgtcggctgcaccgacctgcacgagaacctgggcgtgtgggtggtcttcaagatcaagacgcaggagggctacgcccgcctgggcaacctggagttcatcgaggagaagccgctgatcggcgaggcgctctcccgcgtcaagcgtgcggagaagaagtggcgcgacaagcgcgagaagctccagctggagaccaagcgcgtctacaccgaggccaaggaggccgtggacgccctgttcgtcgactcccagtacgaccagctccaggcggacaccaacatcggcatgatccatgcggctgacaagctggtccaccgcatccgcgaggcgtacctgtccgagctgcccgtcatccctggcgtcaacgcggagatcttcgaggagctggagggccacatcatcaccgccatgtccctctacgacgcgcgcaacgtggtcaagaacggcgacttcaacaacggcctgacgtgctggaacgtcaagggccacgtcgacgtccagcaatcccaccaccgctccgacctggtcatccccgagtgggaggccgaggtgtcccaggccgtccgcgtctgtccgggcaggggctacatcctgcgcgtcaccgcgtacaaggagggctacggcgagggctgcgtcacgatccacgagatcgagaacaacaccgacgagctgaagttcaagaactgcgaggaggaggaggtctacccgacggacaccggcacgtgcaacgactacaccgcgcaccagggcaccgctgcctgcaactcccgcaacgctggctacgaggacgcctacgaggtcgacaccaccgcctccgtcaactacaagccgacctacgaggaggagacctacaccgacgtccgtcgcgacaaccactgcgagtacgaccgcggctacgtgaactacccacccgtccccgctggctacgtcacgaaggagctggagtacttccccgagaccgacaccgtctggatcgagatcggcgagacggagggcaagttcatcgtcgactccgtcgagctgctcctgatggaggagtgatagaattctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaattagatctgtgtgtgttttttg

SEQ ID NO: 12 - protein, 1230 aa, Artificial Sequence; identical to SEQ ID NO: 4
  fully synthetic expression cassette

SEQ ID NO: 13 - DNA, 5600 bp, Artificial Sequence
  fully synthetic expression cassette
gcggccgcgttaacaagcttctgcaggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatcctctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaacctctagaggatcagcatggcgcccaccgtgatgatggcctcgtcggccaccgccgtcgctccgttcctggggctcaagtccaccgccagcctccccgtcgcccgccgctcctccagaagcctcggcaacgtcagcaacggcggaaggatccggtgcatgcaggtaacaaatgcatcctagctagtagttctttgcattgcagcagctgcagctagcgagttagtaataggaagggaactgatgatccatgcatggactgatgtgtgttgcccatcccatcccatcccatttcccaaacgaaccgaaaacaccgtactacgtgcaggtgtggccctacggcaacaagaagttcgagacgctgtcgtacctgccgccgctgtcgaccggcgggcgcatccgctgcatgcaggccatggccacctccaaccgcaagaacgagaatgagatcatcaacgccctgtcgatccccacggtctcgaacccgtccacccaaatgaacctgtccccggacgcccgcatcgaggactccctgtgcgtcgcggaggtcaacaacatcgaccccttcgtctccgcctccacggtccagacgggcatcaacatcgctggccgcatcctcggcgtcctgggcgtcccgttcgctggccagctggcctccttctactccttcctggtcggggagctgtggccctccggtcgcgacccctgggagatcttcctggagcacgtcgagcagctcatccgccagcaagtcaccgagaacacccgcaacacggccatcgcccgcctggagggcctgggccgtggctaccgctcctaccagcaggccctggagacctggctggacaaccgcaacgacgcacgctcccgctccatcatcctggagcgctacgtggcgctggagctggacatcaccaccgccatcccgctcttccgcatccgcaatgaagaggtgcccctgctcatggtctacgcccaggctgccaacctgcacctgctcctgcttcgcgatgcatccctgttcggctccgagtggggcatggcctcgtccgacgtcaaccagtactatcaggagcagatccgctacaccgaggagtactccaaccactgcgtccagtggtacaacaccggcctcaacaacctgcgcggcacgaacgctgagtcctggctgcgctacaaccagttccgccgcgacctgacgctgggcgtcctggacctggtcgccctcttcccctcctacgacacccgcacctaccccatcaacacgtccgcccagctgacccgcgagatctacaccgaccccatcggccgcaccaacgctccctccggcttcgcgtccacgaactggttcaacaacaatgccccgtcgttctccgccatcgaggctgcgatcttccgcccaccgcacctcctggacttccccgagcagctgaccatctactccgcctcgtcccgctggtcgtccacccagcacatgaactactgggtgggccaccgcctcaacttcaggcccatcggtggcaccctgaacacctccacccagggcctgaccaacaacacctccatcaaccccgtcaccctccagttcacgtcccgcgacgtctaccgcaccgagtccaacgccggcaccaacatcctcttcacgaccccggtcaacggcgtcccctgggctcgcttcaacttcatcaacccgcagaacatctacgagcgtggtgcgaccacctactcccagccgtaccagggcgtcggcatccagctcttcgactccgagaccgagctgccacccgagacgaccgagcgtcccaactacgagtcctactcccaccgcctgtcccacatcggcctgatcatcggcaacaccctcagggctcccgtctactcctggacgcaccgctccgcggaccgcacgaacacgatcggtcccaaccgcatcacccagatccccctggtcaaggccctcaacctgcactccggcgtcaccgtcgtgggtggcccaggcttcaccggtggcgacatcctgcgcaggaccaacacgggcaccttcggcgacatccgcctcaacatcaacgtcccgctgtcccagcgctaccgcgtccgcatccgctacgcctccacgaccgacctccagttcttcacgcgcatcaacggcaccacggtcaacatcggcaacttctcccgcaccatgaacaggggcgacaacctggagtaccgctccttccgcaccgccggcttctccaccccgttcaacttcctcaacgcccagtccaccttcacccttggtgcgcagtccttctccaaccaggaggtctacatcgaccgcgtcgagttcgtcccagccgaggtcaccttcgaggccgagtacgacctggagcgtgcccagaaggcggtgaacgccctgttcacctccaccaaccccaggcgcctgaagaccgacgtcacggactaccacatcgaccaggtgtccaacatggtggcctgcctctccgacgagttctgcctggacgagaagcgcgagctgttcgagaaggtcaagtacgcgaagcgcctctccgacgagcgcaacctgctccaggacccgaacttcaccttcatctccggccagctgtccttcgcgtccatcgacggccagtccaacttcccctccatcaacgagctgtccgagcacggctggtggggctccgcgaacgtcaccatccaggagggcaacgacgtcttcaaggagaactacgtcaccctgccgggcaccttcaacgagtgctacccgaactacctctaccagaagatcggcgagtccgagctgaaggcctacacccgctaccagctgcgcggctacatcgaggactcccaggacctggagatctacctcatccgctacaacgcgaagcacgagaccctggacgtccctggcacggactccctgtggcccctctccgtcgagtcgcccatcggccgctgcggcgagcccaaccgctgcgctccccacttcgagtggaaccccgacctggactgctcctgccgcgacggcgagcgctgcgcgcaccattcccatcacttcaccctggacatcgacgtcggctgcaccgacctgcacgagaacctgggcgtgtgggtggtcttcaagatcaagacgcaggagggctacgcccgcctgggcaacctggagttcatcgaggagaagccgctgatcggcgaggcgctctcccgcgtcaagcgtgcggagaagaagtggcgcgacaagcgcgagaagctccagctggagaccaagcgcgtctacaccgaggccaaggaggccgtggacgccctgttcgtcgactcccagtacgaccagctccaggcggacaccaacatcggcatgatccatgcggctgacaagctggtccaccgcatccgcgaggcgtacctgtccgagctgcccgtcatccctggcgtcaacgcggagatcttcgaggagctggagggccacatcatcaccgccatgtccctctacgacgcgcgcaacgtggtcaagaacggcgacttcaacaacggcctgacgtgctggaacgtcaagggccacgtcgacgtccagcaatcccaccaccgctccgacctggtcatccccgagtgggaggccgaggtgtcccaggccgtccgcgtctgtccgggcaggggctacatcctgcgcgtcaccgcgtacaaggagggctacggcgagggctgcgtcacgatccacgagatcgagaacaacaccgacgagctgaagttcaagaactgcgaggaggaggaggtctacccgacggacaccggcacgtgcaacgactacaccgcgcaccagggcaccgctgcctgcaactcccgcaacgctggctacgaggacgcctacgaggtcgacaccaccgcctccgtcaactacaagccgacctacgaggaggagacctacaccgacgtccgtcgcgacaaccactgcgagtacgaccgcggctacgtgaactacccacccgtccccgctggctacgtcacgaaggagctggagtacttccccgagaccgacaccgtctggatcgagatcggcgagacggagggcaagttcatcgtcgactccgtcgagctgctcctgatggaggagtgatagaattctaaatcttattattatcatcgtcgtcgtcgtctcgtcacggaattaattaaagtacctactccgtacttagctagctacaataataaggattcattgatcactacaagagtgatcgactcgactgtagtatgtgtgtgcaatataatgtgctgtctatcaacaactactagtattgtcatttttttcgaaccagggaactttttaatgataagaagaaaaagacaagtacttattgtcgagcatgcgt

SEQ ID NO: 14 - protein, 1230 aa, Artificial Sequence; identical to SEQ ID NO: 4
  fully synthetic expression cassette

(19) United States
(12) Patent Application Publication
(10) Pub. No.: US 2006/0112447 A1
(43) Pub. Date: May 25, 2006

(54) Nucleotide sequences encoding cry1bb proteins for enhanced expression in plants

(76) Inventors: Natalia N. Bogdanova, ST. Louis, MO (US); Charles P. Romano, Chesterfield, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 10/525,318

(22) PCT Filed: Aug. 26, 2003
(86) PCT No.: PCT/US03/26510

§ 371 Date: Oct. 7, 2005

Related U.S. Application Data
(60) Provisional application No. 60/407,428, filed on Aug. 29, 2002.

(51) Int. Cl. A01H 5/00; C12N 15/82; C12N 5/04; A01H 1/00

(52) U.S. Cl. 800/279; 435/468; 800/320.1; 435/412

(57) ABSTRACT

The present invention describes compositions and methods that are useful in the control of lepidopteran insect pests, and more particularly describes nucleotide sequences for use in plants that encode full-length and truncated insecticidal toxins, as well as chimeric toxins. The nucleotide sequences of the present invention exhibit modifications that, when compared to the native sequences obtained from Bacillus thuringiensis species, make them particularly useful for enhanced, improved, and or optimized expression in monocot and dicot plant species. Using methods well known to those skilled in the art the nucleotide sequences described herein can be used to transform plant cells and plant tissue in order to produce transgenic plants that express the encoded proteins, therefore conferring upon the transgenic plants the ability to resist insect infestation.

### 1.0 BACKGROUND OF THE INVENTION

1.1 Field of the Invention

The present invention relates generally to transgenic plants exhibiting insecticidal activity, and to DNA constructs containing genes encoding Cry1Bb proteins for conferring insect resistance when expressed in plants. More specifically, the present invention relates to a method of expressing at least one insecticidal protein in a plant transformed with a gene encoding an insecticidal fragment of a B. thuringiensis δ-endotoxin, resulting in effective control of susceptible target pests.

1.2 Description of Related Art

1.2.1 Methods of Controlling Insect Infestation in Plants The Gram-positive soil bacterium B. thuringiensis is well known for its production of proteinaceous parasporal crystals, or δ-endotoxins, that are toxic to a variety of lepidopteran, Coleopteran, and Dipteran larvae. During the sporulation phase of growth, B. thuringiensis produces crystal proteins that are each specifically toxic to certain species of insects. Many different strains of B. thuringiensis have been shown to produce insecticidal crystal proteins. Compositions comprising B. thuringiensis strains that produce proteins exhibiting insecticidal activity have been used commercially as environmentally acceptable topical insecticides because of their toxicity to the specific target insect pests, and non-toxicity to plants and other non-targeted organisms.

δ-endotoxin crystals are toxic to insect larvae upon ingestion of the crystalline protein composition. Solubilization of the crystal in the alkaline midgut of the insect releases the protoxin form of the δ-endotoxin that, in most instances and particularly for Cry1 type toxins, is subsequently processed to an active toxin by one or more midgut proteases. The activated toxins recognize and bind to the brush-border of the insect midgut epithelium through receptor proteins. Several putative crystal protein receptors have been isolated from certain insect larvae (Knight et al. 1994, Mol. Microbiol. 11:429-436; Gill et al. 1995, Molecular action of insecticides on ion channels, pp. 308-319, Clark, J. M. Editor; Masson et al. 1995, J. Biol. Chem. 270:11887-11896). The binding of active toxins is followed by intercalation and aggregation of toxin molecules to form pores within the midgut epithelium. This process leads to osmotic imbalance, swelling, lysis of the cells lining the midgut epithelium, and eventual larvae mortality.

1.2.2 Transgenic B. thuringiensis δ-Endotoxins as Biopesticides

Plant resistance and biological control are central tactics of control in the majority of insecticide improvement programs applied to the most diverse crops. With the advent of molecular genetic techniques, various δ-endotoxin genes have been isolated and their DNA sequences determined. These genes have been used to construct certain genetically engineered B. thuringiensis products that have been approved for commercial use. Recent developments have seen new &endotoxin delivery systems developed, including plants that contain and express genetically engineered δ-endotoxin genes. Expression of B. thuringiensis δ-endotoxins in plants holds the potential for effective management of plant pests so long as certain problems can be overcome. These problems include the development of insect resistance to the particular Cry protein expressed in the plant, expression in the same plant of two or more insecticidally active proteins toxic to the same insect species and each exhibiting different modes of action, and the presence of the transgene or other elements within the expression cassette in which the transgene resides causing commercially unacceptable morphologies in the transgenic selected events.

Expression of B. thuringiensis δ-endotoxins in transgenic cotton, corn, and potatoes has proven to be an effective means of controlling agriculturally important insect pests (Perlak et al. 1990, BioTechnology 8:939-943; Perlak et al. 1993, Plant Mol. Biol. 22:313-321). Transgenic crops expressing B. thuringiensis δ-endotoxins enable growers to significantly reduce the application of costly, toxic, and sometimes ineffective topical chemical insecticides. Use of transgenes encoding B. thuringiensis δ-endotoxins is particularly advantageous when insertion of the transgene has no negative effect on the yield of desired product from the transformed plants. Yields from crop plants expressing certain B. thuringiensis δ-endotoxins such as Cry1A or Cry3A have been observed to be equivalent to or better than otherwise similar non-transgenic commercial plant varieties. This indicates that expression of some B. thuringiensis δ-endotoxins does not have a significant negative impact on plant growth or development. This is not the case, however, for all B. thuringiensis δ-endotoxins that may be used for expression in plants.

The use of topical B. thuringiensis-derived insecticides may also result in the development of insect strains resistant to the insecticides. Resistance to Cry1A B. thuringiensis δ-endotoxins applied as foliar sprays has evolved in at least one well-documented instance (Shelton et al., 1993, J. Econ. Entomol. 86:697-705). It is expected that insects may similarly develop resistance to B. thuringiensis δ-endotoxins expressed in transgenic plants. Such resistance, should it become widespread, would clearly limit the commercial value of corn, cotton, potato, and other germplasm containing genes encoding B. thuringiensis δ-endotoxins. One possible way to coordinately increase the effectiveness of the insecticide against target pests and to reduce the development of insecticide-resistant pests would be to ensure that transgenic crops express high levels of B. thuringiensis δ-endotoxins (McGaughey and Whalon 1993, Science 258:1451-55; Roush 1994, BioControl Sci. Technol. 4:501-516).

In addition to producing a transgenic plant that expresses B. thuringiensis δ-endotoxins at high levels, commercially viable B. thuringiensis genes must satisfy several additional criteria. For instance, expression of these genes in transgenic crop plants must not reduce the vigor, viability or fertility of the plants, nor should it affect the normal plant morphology. Such detrimental effects have undesired results: they may interfere with the recovery and propagation of transgenic plants; they may also impede the development of mature plants, or confer unacceptable agronomic characteristics.

There remains a need for compositions and methods useful in producing transgenic plants that express B. thuringiensis δ-endotoxins at levels high enough to effectively control target plant insect pests as well as prevent the development of insecticide-resistant pest strains. A method resulting in higher levels of expression of the B. thuringiensis δ-endotoxins will also provide the advantages of more frequent attainment of commercially viable transformed plant lines and more effective protection from infestation for the entire growing season.

There also remains a need for a method of increasing the level of in planta expression of B. thuringiensis δ-endotoxins that does not simultaneously result in plant morphological changes that interfere with optimal growth and development of desired plant tissues. For example, the method of potentiating expression of the B. thuringiensis δ-endotoxins in maize should not result in a corn plant which cannot optimally develop for cultivation and harvest of the crop.

Additionally, there remains a need for compositions and methods useful in producing transgenic plants which express two or more Bacillus thuringiensis δ-endotoxins toxic to the same insect species and which confers a level of resistance management for delaying the onset of resistance of any particular susceptible insect species to one or more of the insecticidal agents expressed within the transgenic plant. Alternatively, expression of a Bacillus thuringiensis insecticidal protein toxic to a particular target insect pest along with a different proteinaceous agent toxic to the same insect pest but which confers toxicity by a means different from that exhibited by the Bacillus thuringiensis toxin is desirable. Such other different proteinaceous agents comprise Xenorhabdus sp. or Photorhabdus sp. insecticidal proteins, deallergenized and de-glycosylated patatin proteins or permuteins thereof, Bacillus thuringiensis vegetative insecticidal proteins, lectins, and the like. One means for achieving this result would be to produce two different transgenic events, each event expressing a different insecticidal protein, and breeding the two traits together into a hybrid plant. Another means for achieving this result would be to produce a single transgenic event expressing both insecticidal genes. This can be accomplished by transformation with a nucleotide sequence that encodes both insecticide proteins, but another means would be to produce a single event that was transformed to express a first insecticide gene, and then transform that event to produce a progeny event that expresses both the first and the second insecticide genes.

Achievement of these goals such as sufficient co-expression of multiple insecticidally active proteins in the same plant, and/or high expression levels of insecticidal proteins which do not result in aberrant morphological effects upon the transgenic plant has been elusive, and their pursuit has been an ongoing and important aspect of the long term value of insecticidal plant products.

More than two-hundred and fifty individual insecticidal proteins have been identified from Bacillus thuringiensis species, but only a handful of these have been tested for expression in plants. Initially, the native sequences were utilized in plant expression cassettes, and these proved useless for producing transgenic plants exhibiting insecticidal properties. This was likely due to the fact that native Bacillus thuringiensis nucleotide sequences exhibit a nucleotide composition substantially different from that in plants. Modifications to sequences encoding Bacillus thuringiensis toxin proteins which substantially reduces the AT nucleotide composition results in substantial improvements in levels of expression of some of these proteins in plants, however, expression of Bacillus thuringiensis δ-endotoxins in plants is not without effect. It requires trial and error experimentation to determine which if any Bacillus thuringiensis δ-endotoxin protein when expressed in planta will produce a commercially useful plant, which exhibits levels of expression that are effective in controlling target insect pests, and which does not result in morphologically abnormal effects upon the plant. Examples of Bt proteins that have been successfully expressed in plants are substantially limited to Cry1Ab, Cry1Ac, Cry2Ab, amino acid sequence variants of Cry3Bb, Cry1C, and Cry3C. Cry2Ab was only successfully expressed when targeted for importation into chloroplasts. Cry1 proteins have been expressed in plants as full-length protoxins exhibiting an amino acid sequence that is substantially similar to the form in which they are found in nature when expressed by Bacillus thuringiensis species. Cry1 proteins have also been expressed in plants as less than full-length forms of the protein, comprising essentially the tryptic core or active toxin domain of the Cry1 protein. However, Cry1 proteins have not been expressed at high levels. Since the majority of acreage planted on an annual basis with recombinant plants exhibiting insecticidal bioactivity consists substantially of plants expressing Cry1A proteins, the likelihood of the onset of resistance to Cry1A proteins by target insect pest species is greater than it would be if a second mode of action of insect control was also packaged in some way or expressed along with the cry1 allele, or if the cry1 allele was expressed at high levels.

To date, no field resistance has been observed. However, there have been several examples of acquired resistance to Cry1A proteins under laboratory conditions. Therefore, it is imperative that plants currently expressing only one Cry protein be replaced with plants containing additional genes encoding insecticidal proteins exhibiting different mechanisms of insecticidal activity. Thus, the discovery of new Bacillus thuringiensis isolates and new uses of known Bacillus thuringiensis isolates remains an empirical and unpredictable art. There also remains a need for new toxin genes that can be expressed at adequate levels in plants in a manner that will result in the effective control of target insect pest species.

### 2.0 SUMMARY OF THE INVENTION

The present invention provides compositions and methods for use in controlling target insect pests, and in particular lepidopteran insect pest species susceptible to Cry1Bb insecticidal crystal proteins or insecticidal variants thereof. More specifically the subject invention provides expression cassettes for use in plants, the expression cassettes containing at least nucleotide sequences encoding the full length Cry1Bb protein, or variants thereof, which exhibit at least the level of insecticidal activity as the native full length Cry1Bb protein, or insecticidally active fragments thereof, which confer insect inhibitory traits to a plant expressing the protein from within the cassette provided. The nucleotide sequences of the present invention encoding Cry1B proteins or insecticidal fragments thereof contain modifications in comparison to the native Bacillus thuringiensis cry1Bb coding sequence which result in improved expression of the Cry1Bb protein in plants compared to expression levels observed in plants using the native Bt cry1Bb coding sequence, and which make these sequences particularly well suited for expression of the Cry1Bb protein in plants.

The invention provides in one embodiment nucleotide sequences exhibiting Cry1Bb variant coding sequences that are optimized for expression in plants to produce an insect inhibitory amount of a Cry1Bb protein or insecticidal fragment thereof which is toxic or inhibitory to one or more target lepidopteran insect pest species. These nucleotide sequences include plant preferred Cry1Bb coding sequences as set forth in SEQ ID NO:3, 5, 8, 11, and 13, or as contained within the vectors or nucleotide sequence fragments corresponding to pMON33733, pMON33734, pMON40227, and pMON40228. Those skilled in the art will recognize that these sequences, in particular the sequences as set forth in the SEQ ID NO's herein, can be artificially synthesized and introduced into any vector of interest for use in expressing the sequences disclosed herein or sequences substantially the same as those set forth herein in plants. Such sequences are prepared by extrapolating a preferred nucleotide sequence from the amino acid sequence desired for expression in plants and producing that nucleotide sequence through any number of means available in the art. The preferred means uses phosphoramidite chemistries to construct short oligonucleotides that are each then linked together for form the full length sequence.

The invention also provides expression cassettes for use in plants containing sequences encoding all of, or an insecticidally active fragment of, or an amino acid sequence variant of, a Cry1Bb protein for use in transforming plants to express said sequences. Nucleotide sequences comprising exemplary expression cassettes are referred to herein and as set forth in SEQ ID NO:5, SEQ ID NO:8, SEQ ID NO:11, and SEQ ID NO:13. The subject invention also provides novel amino acid sequences comprising all or an insecticidally active fragment of a Cry1Bb protein or equivalent as set forth in SEQ ID NO:2, SEQ ID NO:4, SEQ ID NO:7, SEQ ID NO:10, SEQ ID NO:12, and SEQ ID NO:14. A polynucleotide sequence encoding an insecticidal fragment of a Cry1Bb can be selected from the group of sequences consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13. Additionally, sequences encoding the amino acid sequences set forth in SEQ ID NO:2 from about amino acid position 2 through about amino acid position 600, SEQ ID NO:4 from about amino acid position 3 through about amino acid position 601, SEQ ID NO:7 from about amino acid position 3 through about amino acid position 601, SEQ ID NO:10 from about amino acid position 3 through about amino acid position 601, SEQ ID NO:12 from about amino acid position 3 through about amino acid position 601, and SEQ ID NO:14 from about amino acid position 3 through about amino acid position 601 and that hybridize to the range of nucleotide sequences as set forth above under stringent hybridization conditions are within the scope of the present invention and comprise insecticidally active fragments. Indeed, any peptide, for example comprising from about amino acid position 2 through about from as little as amino acid position 600 up through amino acid position 1229 to 1230 as set forth in SEQ ID NO:4 is considered to be within the definition of an insecticidally active fragment. These proteins that are at least from about 598 to about 600 amino acids are sequences that are representative of insecticidal fragments of the full length Cry1Bb insecticidal protein exemplified from about amino acid position 2 through about amino acid position 1228 or 1229 and are considered herein to be within the scope of the present invention.

An additional embodiment consists of breeding together a first transgenic plant transformed to contain a first nucleotide sequence encoding a first Bt insecticidal protein and a first herbicide tolerance marker with a second transgenic plant transformed to contain a second nucleotide sequence different from the first, encoding a second Bt insecticidal protein different from the first, and a second herbicide tolerance marker different from the first, to produce a third transgenic plant comprising a hybrid plant comprising both the first and the second insecticidal proteins and the first and second herbicide tolerance markers. The herbicide tolerance markers are selected from but not limited to the group consisting of a gox enzyme, an antibiotic resistance marker such as nptII, a glyphosate insensitive EPSPS enzyme, a basta resistance marker, and any other herbicide tolerance marker known in the art, for example. The Bt insecticidal proteins can be selected from any of the known Cry1, Cry1, Cry3, Cry4, Cry5, Cry6, Cry9, Cry22, Cry33/34 binary toxins, as well as any other Bt insecticidal proteins known in the art such as VIP proteins and the like. As exemplified herein, the first insecticidal protein may be a Cry1Bb protein toxic to lepidopteran species, and the second insecticidal protein need not be within the class of insecticidal proteins that controls lepidopteran species, but instead can be within the class of proteins known to be toxic to certain coleopteran insect species such as Cry3 proteins, Cry5 proteins, various binary toxins known in the art, VIP proteins, and the like.

In fact, a first insecticidal resistance gene can be transformed into a first plant along with a first selectable marker, such as a herbicide tolerance gene, to produce a first transgenic plant. A second insecticidal resistance gene different from the first can be transformed into a second plant along with a second selectable marker, such as a second herbicide tolerance gene, to produce a second transgenic plant. The first and the second transgenic plants can then be mated, assuming the first and second plants are sufficiently related and capable of being bred together, to produce a hybrid transgenic plant containing both of the transgene alleles of the first transgenic plant and both of the transgene alleles of the second transgenic plant.

Other embodiments of the invention as set forth herein consist of plants comprising the nucleotide sequences as set forth herein, plants comprising nucleotide sequences which are substantially identical to the nucleotide sequences as set forth herein in which the sequence present in plants comprises all or a part of the coding sequence for expression of a Cry1Bb or amino acid sequence variant thereof in plants, said all or part of the coding sequence encoding a Cry1Bb or amino acid sequence variant thereof sufficient to exhibit insecticidal activity to one or more target insect plant pests of corn, cotton or soy and the like and which is no less toxic than the native full length Cry1Bb insecticidal toxin. Plants, plant parts, progeny, and progeny or hybrid plants derived from breeding with the recombinant plants of the present invention are encompassed as well, in particular those plants which contain one or more of the nucleotide sequences of the present invention which encode a Cry1Bb protein or insecticidal portion of said protein. The sequences of the present invention are also intended to include nucleotide sequences exhibiting at least from about 75% to about 99% or greater sequence identity with the sequences of the present invention. In addition, the sequences of the present invention are intended to include sequences that hybridize under stringent conditions to the sequences as set forth in the sequence listing herein.

A plant cell comprising a nucleotide sequence that functions for improved expression in plants compared to a native Bt sequence encoding a Cry1Bb protein or insecticidal fragment thereof is contemplated herein. Such plant cells are transformed with a nucleotide sequence that comprises a sequence selected from but not limited to the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13. Alternatively, a complete Cry1Bb protein sequence can be expressed resulting in a protein exhibiting an amino acid sequence substantially that as set forth in SEQ ID NO:4 from about amino acid position three through about amino acid position 1229 or 1230. A method for preparing a transgenic plant cell as described herein containing a nucleotide sequence encoding a full length Cry1Bb or an insecticidally active fragment thereof is contemplated. Transgenic plants produced from the transformed cells are also within the scope of the present invention. In particular but not intending to be limited by such disclosure, the plants including but not limited to maize, wheat, sorghum, oat, barley, cotton, potato, tomato, soybean, canola, and fruit trees are specifically included within the scope of the present invention. Plants transformed with other nucleotide sequences encoding yet insecticidal proteins other than the insecticidal protein of the present invention (Cry1Bb) can be bred to plants transformed to contain only the Cry1Bb coding sequence, resulting in a third plant that is also a recombinant plant by virtue of it's heritage, and that exhibits improved insect resistance and tolerance to insect infestation as a result of the presence of the two different insecticidal proteins. Furthermore, such progeny of a breeding can be easily and simply identified by ensuring that each parental plant has a selectable marker present for conveying a double selection pressure upon the hybrid plant produced as a result of the breeding of the two or more plants. The result of course is a hybrid recombinant plant tat exhibits at least one type of insect resistance (for example, a first insect resistance conveyed by the Cry1Bb gene, resistance to lepidopteran pests) but which may also exhibit a different insect resistance to the same insect pests controlled by the Cry1Bb (which may be one or more of an insecticidal protein including but not limited to a Cry1, a Cry2, a Cry4, a Cry5, a Cry6, a Cry9, and a VIP1, VIP2, or a VIP3) or which may exhibit a resistance to an entirely different class of plant insect pest species such as to Coleopteran species (which may require the use of one or more of a Cry3A, a Cry3B, a Cry3C, a Cry22, ET70, TIC851, a binary Bt insecticidal protein toxin such as ET 33/34, ET80/76, or a CryP149B1).

Stringent conditions as defined herein include moderate to high stringency conditions which achieve the same, or about the same, degree of specificity of hybridization as the conditions employed by the applicants as exemplified herein. Examples of moderate and high stringency conditions are provided herein. Specifically, hybridization of immobilized nucleotide sequences on means used for Southern blotting or on hybridization chips such as are well known in the art, for example, with ³²P-labeled gene-specific probes or primers can be performed by standard methods (Sambrook, Fritsch, & Maniatis; Molecular Cloning, A Laboratory Manual, 2nd Ed., Cold Spring Harbor Laboratory Press, NY 1989). In general, hybridization and subsequent washes can be carried out under moderate to high stringency conditions that allow for detection of target sequences with homology to the exemplified toxin genes. For double-stranded nucleotide probes, hybridization can be carried out D overnight at 20-25 C below the melting temperature (Tm) of the DNA hybrid in 6×SSPE, 5× denhardts solution, 0.1% SDS, 0.1 mg per ml denatured nucleotide probe. The melting temperature can be described by the following formula as set forth in Beltz et al. (1983, Methods in Enzymology, 100:266-285, Wu, Grossman, and Moldave Eds., Academic Press, NY)
Tm=81.5 C+16.6 Log[Na^{+]}+0.41 (% G+C)−0.61 (% formamide)−600/length of duplex in base pairs.
Washes are typically carried out as follows:
(1) two washes at room temperature for about fifteen (15) minutes in 1×SSPE, 0.1% SDS (low stringency wash), followed by
(2) one wash at Tm −20 C for about fifteen (15) minutes in 0.2×SSPE, 0.1% SDS (moderate stringency wash).

For oligonucleotide probes, hybridization can be carried out overnight at 10-20 C below the melting temperature (Tm) of the hybrid in 6×SSPE, 5× Denhardts solution, 0.1% SDS, 0.1 μg per ml denatured probe. The Tm for oligonucleotide probes can be described by the following formula as set forth in Suggs et al. (1981, ICN-UCLA Symp. Dev. Biol. Using Purified Genes, 23:683-693, D. D. Brown Ed., Academic Press, NY):
Tm(C)=2(No. T&A base pairs)+4(No. G&C base pairs).
Washes using oligonucleotide probes can be carried out as described above. For probe sequences of greater than about seventy (70) nucleotides in length, a low stringency condition for hybridization would be equivalent to suspension in either 1× or 2×SSPE at a temperature from about room temperature to about 42 C. A moderate stringency condition for hybridization would be equivalent to suspension in from about 0.2× to about 1×SSPE at a temperature of about 65 C. A high stringency hybridization condition would be equivalent to suspension in from about 0.01× or less to about 0.1×SSPE at a temperature of about 65 C.

The amino acid sequences of the present invention are intended to include analogs or homologs or other related amino acid sequences which are sufficient to exhibit insecticidal bioactivity at least equivalent to that exhibited by the native Cry1Bb full length protein, including at least amino acid sequences which are from about 95% identical to about 99% identical or greater in amino acid sequence to the sequence exhibited by the amino acid sequence as set forth in SEQ ID NO:2 or SEQ ID NO:4.

Another embodiment of the present invention provides a method for transforming a plant to express a Cry1Bb protein or amino acid sequence variant or insecticidally active fragment thereof.

Still another embodiment provides methods for detecting the presence of a sequence disclosed herein in the present invention in a plant, plant cell, or biological sample. The detection of a nucleotide sequence expressing Cry1Bb protein in a plant would be diagnostic for a plant containing said nucleotide sequence within its nuclear or plastid genome. Furthermore, antibodies which specifically bind to a Cry1Bb protein are set forth in the examples. Such antibodies are exemplary for use in detecting the presence of a plant expressing all or a part of a Cry1Bb protein, and for detecting a plant comprising a nucleotide sequence that encodes a Cry1Bb protein. The detection of Cry1Bb protein using immunological methods would be diagnostic for a plant comprising any of the nucleotide sequences set forth herein which express a Cry1Bb protein or equivalent.

A biological sample consisting primarily of a plant containing one or more of the nucleotide sequences of the present invention is believed to be within the scope of the present invention. A biological sample derived from a plant, a plant tissue, or a plant seed, wherein the sample contains a nucleotide sequence that is or is complementary to a sequence selected from but not limited to a group of sequences consisting of SEQ ID NO:3, SEQ ID NO:5, SEQ ID NO:8, SEQ ID NO:11, and SEQ ID NO:13, in which the sequence is detectable in the sample using a nucleic acid amplification or nucleic acid hybridization method, is contemplated specifically herein to be within the scope of the present invention. A biological sample is intended to include a plant, plant tissue, or plant seed that contains one or more of the nucleotide sequences exemplified herein, as well as products produced from such plant, plant parts, or plant seeds including but not limited to flour derived from soy or wheat or barley or oat or potato or corn, soy or corn meal, corn syrup, corn or soy or canola oils, corn starch, and cereals manufactured in whole or in part to contain corn, soy, wheat, barley, oat, flax or other cereal plant by-products that contains a detectable amount of one or more of the nucleotide sequences of the present invention, wherein the nucleotide sequences are detectable in said biological sample or extract using any nucleic acid amplification or nucleic acid hybridization method.

Similarly, a kit for detecting the presence of Cry1Bb protein in a sample is contemplated by the instant invention. The kit would provide a test reagent containing a Cry1Bb positive control sample along with a negative control, antibodies which bind specifically to a Cry1Bb protein, and the reagents necessary to carry out a determinative reaction with the control samples as well as an unknown sample suspected of containing an immunologically detectable amount of a Cry1Bb protein, packaged together in said kit with instructions for use. Antibodies that bind specifically to Cry1Bb and not to other Bt insecticidal proteins are particularly suited for use in kits based on immunological methods and are believed to be within the scope of this invention. A similar kit for detecting the presence of a nucleotide sequence as set forth herein, encoding at least an insecticidally active Cry1Bb protein or fragment thereof, is specifically contemplated herein. Exemplary are nucleotide sequences which could be used as probes for detecting a sufficient amount of a nucleotide sequence derived from a polynucleotide sequence encoding a Cry1Bb protein, or nucleotide sequences in the form of primer pairs which could be used as amplification primers for producing all or a part of the Cry1Bb encoding nucleotide sequences encompassed by this disclosure, for example by using thermal amplification methods well known in the art. Such primers or probes along with positive and negative control samples packaged together in a kit, or packaged separately, and distributed with the necessary reagents for completing a hybridization or amplification reaction to detect all or a part of the Cry1Bb encoding nucleotide sequences encompassed by the instant invention, along with instructions for use are specifically contemplated herein.

The regulation of expression of the sequences of the present invention can be accomplished in a number of different ways. One means would be to rely on the particular operably linked promoter sequence which drives expression of the transgene to effectively regulate the expression of the Cry1Bb protein. Generally, this means results in the expression being determined by the type of linked promoter, i.e., a promoter that is temporally or spatially regulated within the cell or tissue type within the plant by factors that are beyond the control of the skilled artisan. Promoters such as these are generally either “on” at all times throughout the growth and development of the plant. Other promoters may be “enhanced” in that they are on at characteristically prominent times, for example, only when the plant is flowering, or only when the plant is developing from an embryo within the germinating seed into a shoot or a hairy root, or only substantially within the root, etc. The range of promoters available for such temporal and spatial expression within a plant, and more particularly, within a plant type, is too numerous to discuss here. However, using antisense technologies, the transcribed messenger RNA can be regulated in such a way as to elevate the level of protein produced within a plant or to decrease the level of protein produced in a plant. One particularly useful means for regulating the level of messenger RNA in a cell is RNAi technology exemplified in WO 01/75164 (Tuschl et al.), WO 99/61631 (Heifetz et al.), WO 99/53050 (Waterhouse et al.), WO 99/49029 (Graham et al.), WO 99/32619 (Fire et al.), WO 98/05770 (Werner et al.). A summary of the known RNAi technology can be found at Lau et al. Scientific American August 2003 pp. 3441). The expression of the constructs exemplified herein in plants can be subjected to these means for regulating and modulating the expression of the proteins expressed therefrom.

### 3.0 DESCRIPTION OF THE SEQUENCES

SEQ ID NO:1 represents a native Bacillus thuringiensis nucleotide sequence encoding a native Cry1Bb protein as set forth in Donovan et al., U.S. Pat. No. 5,679,343, and described therein as cryET5 encoding CryET5.

SEQ ID NO: represents the deduced full length amino acid sequence translation of a native Cry1Bb protein from the open reading frame identified as being present in the nucleotide sequence of SEQ ID NO:1.

SEQ ID NO:3 represents a non-naturally occurring or synthetic nucleotide sequence exhibiting, when compared to the native coding sequence, improved in planta levels of expression of a Cry1Bb variant protein, and which encodes an amino acid sequence variant of a Cry1Bb protein. SEQ ID NO:4 represents the deduced amino acid sequence translation of the nucleotide sequence as set forth in SEQ ID NO:3 encoding a Cry1Bb amino acid sequence variant.

SEQ ID NO:5 represents a non-naturally occurring nucleotide sequence comprising an expression cassette comprising the operably linked elements P-FMV: L-Os.βtub : I-Os.PAL: cry1Bb1 variant: T-Os.Ldh (corresponding to a figwort mosaic virus promoter, a rice pal gene intron, a synthetic nucleotide sequence encoding a Cry1Bb variant protein, and a rice lactate dehydrogenase termination and polyadenylation sequence) present as set forth in both pMON33731 and pMON33733, exhibiting improved in planta levels of expression of a Cry1Bb variant protein.

SEQ ID NO:6 represents the amino acid sequence translation of a nucleotide sequence as set forth in SEQ ID NO:5 from about nucleotide position 526 through about nucleotide position 1317 encoding an NptII protein used primarily in the applications as set forth herein as a selectable marker for identifying plant cells and plants transformed by a vector or sequence containing the nptII gene linked to some other gene of interest.

SEQ ID NO:7 represents the amino acid sequence translation of a nucleotide sequence as set forth in SEQ ID NO:5 from about nucleotide position 2644 through about nucleotide position 6333 encoding a Cry1Bb amino acid sequence variant.

SEQ ID NO:8 represents a non-naturally occurring or synthetic nucleotide sequence comprising an expression cassette comprising the operably linked elements P-FMV: L-Os.βtub : I-Os.PAL : TP-Zm.rbcs: cry1Bb1 variant: T-Os.Ldh (corresponding to the following operably linked genetic elements: a figwort mosaic virus promoter, a rice pal gene intron sequence, a sequence encoding a corn or maize ribulose bis-phosphate carboxylase synthase small subunit chloroplast targeting peptide (rbcs) interrupted by a small intron native to the corn sequence, a coding sequence encoding a Cry1Bb amino acid sequence variant, and a rice lactate dehydrogenase transcription termination and polyadenylation sequence) present in both pMON33732, pMON33734, pMON33750, and pMON40213 (except that a sequence encoding a glyphosate tolerant CP4 EPSPS is present in place of the NptII coding sequence in pMON33750 and pMON40213) that exhibits enhanced in planta expression of the plastid targeted Cry1Bb amino acid sequence variant.

SEQ ID NO:9 represents the amino acid sequence translation of a nucleotide sequence as set forth in SEQ ID NO:8 from about nucleotide position 526 to about nucleotide position 1317 encoding an NptII protein used primarily in the applications as set forth herein as a selectable marker for identifying plant cells and plants transformed by a vector or sequence containing the nptII gene linked to some other gene of interest.

SEQ ID NO:10 represents the amino acid sequence translation of the nucleotide sequence as set forth in SEQ ID NO:8 from about nucleotide position 3041 through about 6730 encoding a plastid targeted Cry1Bb amino acid sequence variant.

SEQ ID NO:11 represents a non-naturally occurring or synthetic nucleotide sequence comprising an expression cassette comprising the operably linked elements P-e35S : L-TaCab : 1-Os.Act1 : cry1Bb1 variant : T-Ta.Hsp17 (corresponding to the following operably linked elements: enhanced cauliflower mosaic virus ³⁵S promoter, a 5′ untranslated wheat chlorophyll a/b binding protein gene leader sequence, a rice actin intron sequence, a Cry1Bb amino acid sequence variant coding sequence, and a wheat hsp17 heat shock gene transcription termination and polyadenylation sequence) present in pMON40227 exhibiting enhanced in planta expression of a Cry1Bb amino acid sequence variant.

SEQ ID NO:12 represents the amino acid sequence translation of the nucleotide sequence as set forth in SEQ ID NO:11 from about nucleotide position 1241 through about nucleotide position 4930 encoding a Cry1Bb amino acid sequence variant.

SEQ ID NO:13 represents a non-naturally occurring nucleotide sequence comprising an expression cassette comprising the operably linked elements P-e35S : L-Ta.Cab : I-Os.Act1: TP-Zm.rbcs: cry1Bb1 variant : T-Ta.Hsp17 (corresponding to the following operably linked elements: enhanced cauliflower mosaic virus promoter, a 5′ untranslated wheat chlorophyll a/b binding protein gene leader sequence, a rice actin intron sequence, a sequence encoding a corn or maize ribulose bis-phosphate carboxylase synthase small subunit chloroplast targeting peptide (rbcs) interrupted by a small intron native to the corn sequence, a synthetic sequence encoding a Cry1Bb1 amino acid sequence variant, and a wheat heat shock Hsp17 protein transcription termination and polyadenylation sequence) present in pMON40228 exhibiting improved in planta expression of a plastid targeted Cry1Bb amino acid sequence variant.

SEQ ID NO:14 represents the amino acid sequence translation of the nucleotide sequence as set forth in SEQ ID NO:13 from about nucleotide position 1652 through about nucleotide position 5341 encoding a plastid targeted Cry1Bb amino acid sequence variant.

### 4.0 DETAILED DESCRIPTION OF THE INVENTION

The subject matter encompassed by the instant invention includes compositions and methods for use in the control of plant infestation by insect pest species, and in particular, control of infestation by larvae of various lepidopteran insect pest species susceptible to or controlled by ingestion of insecticidally effective amounts of a Bacillus thuringiensis Cry1Bb protein. More specifically, nucleotide sequences which have been designed for enhanced and/or improved expression of Cry1Bb pesticidal toxin in plant cells and in plant tissue are encompassed by the instant invention, including full length Cry1Bb, core toxin or tryptic fragments of Cry1Bb, less than full length Cry1Bb toxin, and fragments which are smaller in mass than the core or tryptic fragment but which retain insecticidal bioactivity to one or more insect species which are normally inhibited or killed by ingestion of full length Cry1Bb toxin.

Reference to “full length” is intended to include but is not intended to be limited to a nucleotide sequence which encodes all of the native Cry1Bb toxin or an amino acid sequence variant of the Cry1Bb toxin which retains bioactivity no less than that observed for controlling at least one insect pest species normally controlled by the native Cry1Bb toxin. The term “full length” is also intended to refer to the form of the Cry1Bb toxin produced or expressed from a nucleotide coding sequence of the instant invention. A full length Cry1Bb toxin protein will be recognized by one skilled in the art to be a protein substantially identical in length of amino acid sequence to the native Cry1Bb protein expressed from the native gene in Bacillus thuringiensis. A typical Cry1 protein is comprised of a toxin domain positioned at the amino terminal end of the Cry1 protein sequence and a protoxin domain linked to and positioned at the carboxy-terminal end of the toxin domain. The toxin domain is typically further comprised of three sub-domains described in the literature as domain I, domain II, and domain III, the precise location of the region defining either end of each of these sub-domains being somewhat arbitrary but generally based on degrees of homology, identity, or similarity between amino acid sequences of other Cry1 proteins within a particular class of Cry1's. Generally, domain I is positioned at the amino terminal end of the toxin domain and is linked at its carboxy terminal end to the amino terminal end of domain II, which is in turn linked at its carboxy terminal end to the amino terminal end of domain III. Sub-domains of the toxin domain have also been identified in the art by reference to amino acid sequence position along the length of a given Cry1 protein. Interestingly, Cry2 and Cry3 toxin proteins exhibit this structural similarity, although the degree of identity between sub-domains when comparing Cry1's to either Cry2 or Cry3 proteins is more divergent. An insecticidal fragment of any of the proteins of the present invention will be recognized by those of skill in the art as any amino acid sequence which is greater than about 95% identical at the amino acid sequence level to the Cry1Bb proteins of the present invention and which retain insecticidal bioactivity no less than that of the full length Cry1Bb1 (CryET5) native protein. Preferred insecticidal fragments of the present invention include from about amino acid sequence position one through about amino acid position 600, or through about amino acid position 643, or of the sequences as set forth in either SEQ ID NO:2 or SEQ ID NO:4, or amino acid sequences which are substantially the same as those sequences or within a range of about 95% sequence identity at the amino acid sequence level to the amino acid sequence of the first 643 or so amino terminal amino acids.

A number of insecticidally useful chimeric proteins have been disclosed which are comprised of combinations of sub-domains from different Bacillus thuringiensis insecticidal crystal protein toxins. For example, Fischhoff et al. described a chimeric toxin formed from linking domains I and II of a first Cry protein, Cry1Ab, to domain III of a second Cry protein, Cry1Ac, which exhibited insecticidal bioactivity at least as great as the insecticidal bioactivity of either of the parent toxins (U.S. Pat. Nos. 5,500,365, 5,880,275). Perlak et al. also described a gene identical to that of Fischhoff et al. (BioTechnol. 1990, 8:939-943). Bosch et al. also disclosed chimeric toxins comprising a variety of formulations consisting of domains I and II of a first Cry protein linked to domain III of a Cry protein different from the first, and noted that it was unpredictable to determine which, if any, would function in providing insecticidal activity at least as great as that of the parent toxins (WO95/06730). Malvar et al. have also disclosed chimeric amino acid sequences formed from the operable linkage, from amino to carboxy terminal ends, of domain I of a first Cry protein with domain II and domain III of a second Cry protein which is different from the first Cry protein; and domain I and domain II of a first Cry protein with domain III of a second Cry protein which is different from the first (U.S. Pat. Nos. 6,017,534, 6,110,464, 6,221,649, and 6,242,241). It is likely that other such chimeric toxins could also be constructed, but it would not be known which if any of the chimeric toxins would exhibit insecticidal activity, and whether any insecticidal activity would be an improvement over any of the native toxins from which the sub-domains were selected for incorporation into the chimera.

The nucleotide sequences of the present invention exhibit individual nucleotides and sequences of nucleotides that are different in composition relative to the corresponding coding sequences contained within the native Bacillus thuringiensis sequence encoding Cry1Bb. Such differences include reductions in the overall adenosine and thymidine composition of the nucleotide sequence compared to the native Bt sequence; a modified preference for various codons which, in Bacillus thuringiensis, would otherwise be preferred for use, in particular with reference to the third base position for each codon such that for amino acids for which there are at least two or more codons, a preference for use of those codons which do not have an A or a T in the third base position; and an overall guanosine and cytosine composition from about 50% to about 60% or more; and an overall reduction in the appearance of putative polyadenylation sequences as set forth in Fischhoff et al. (U.S. Pat. No. 5,500,365). Such nucleotide sequences of the present invention which encode all or an insecticidally active fragment of a Cry1Bb protein exhibit an improved level of expression in plants compared to the native Cry1Bb protein sequence obtained from Bacillus thuringiensis, particularly when operably linked at least to a plant functional promoter and a plant functional transcription termination and polyadenylation sequence, or when operably linked to a promoter functional in a plant chloroplast and targeted for expression within the plant chloroplast. The sequences of the present invention are therefore particularly well-suited for optimized expression in plants, and can be used by those skilled in the art to transform plant cells, regenerate recombinant plants from the transformed plant cells, and to obtain commercially useful plants which express insecticidally effective amounts of all or an insecticidally active fragment of a Cry1Bb protein for inhibiting insect infestation of the plant. The words “plant functional”, with reference to nucleotide sequences, are intended to indicate that the particular sequence referred to, such as a promoter, an intron, an untranslated leader, a transcription initiation sequence, a coding sequence, and/or a transcription termination and polyadenylation sequence operates in a plant with the molecular and cellular machinery involved in transcription and translation and post translation in a way which is intended to bring about the production of an amino acid sequence encoded by the coding sequences to which the plant functional sequences are linked.

In one embodiment, the invention provides nucleotide sequences for expression in plants that encode a Cry1Bb toxin or an insecticidally active fragment of a Cry1Bb toxin that is active against lepidopteran insects. These nucleotide sequences include genes designed for expression in plants, and these genes can be selected from the group consisting of SEQ ID NO:3, SEQ ID NO:5, SEQ ID NO:8, SEQ ID NO:11, and SEQ ID NO:13.

In another embodiment, the invention also provides nucleotide sequences for expression in plants that encode a Cry1Bb protein or fragment thereof toxic to lepidopteran insect pests that typically infest commercial crops. Such protein sequences include SEQ ID NO:2, SEQ ID NO:4, SEQ ID NO:7, SEQ ID NO:10, SEQ ID NO:12, and SEQ ID. NO:14. Pests typically infesting commercial crops are described herein, but include at least armyworms, rootworms, boll worms, loopers, earworms, bud worms, and stem borers.

The subject invention provides nucleotide sequences encoding an insecticidally active fragment of a Cry1Bb protein linked to a protoxin domain of a Cry1 toxin other than a Cry1Bb toxin. Conversely, the present invention also provides a novel nucleotide sequence encoding a Cry1Bb protoxin domain which can be used for constructing a nucleotide sequence encoding a full length Cry1 related toxin in which the toxin domain is other than a Cry1Bb toxin domain. Additionally, the present invention provides nucleotide sequences encoding amino acid sequences corresponding to sub-domains of a Cry1Bb toxin fragment, and more particularly corresponding to domain I, domain II, and domain III of the Cry1Bb toxin fragment, which can be used to construct novel toxins comprising all or any part of each of these sub-domains of the Cry1Bb toxin domain amino acid sequences.

In another embodiment, the present invention provides nucleotide sequences that express a Cry1Bb toxin that is less than full length compared to the full length Cry1Bb toxin produced by Bacillus thuringiensis. Such nucleotide sequences encoding a less than full length Cry1Bb amino acid sequence typically do not contain all or a portion of the protoxin fragment of the full-length native Cry1Bb protein. Nucleotide sequences encoding a less than full-length Cry1Bb amino acid sequence could be used for the production of nucleotide sequences which encode a fusion or chimeric protein toxin.

One example of a nucleotide sequence which has been designed for enhanced and/or improved expression of Cry1Bb pesticidal toxin in plant cells and in plant tissue is SEQ ID NO:3 which substantially encodes a native Cry1Bb amino acid sequence. The difference between the amino acid sequence encoded by SEQ ID NO:3 and the native Cry1Bb sequence resides in the amino terminus of the peptide sequence. The native coding sequence (SEQ ID NO:1) initiates with the codon “ttg”, which upon translation of the corresponding position in the mRNA corresponding to the transcription product produced from the cry1Bb gene in Bacillus thuringiensis results in the incorporation of a leucine amino acid residue at the first amino acid sequence position in the native Cry1Bb protein (SEQ ID NO:2 herein, and referenced in Donovan et al., U.S. Pat. No. 5,679,343). The second and third amino acid residues comprising the native Cry1Bb sequence are threonine and serine respectively. While the plant functional coding sequences of the present invention encode an amino acid sequence identical to the composition of the native Cry1Bb amino acid sequence corresponding to the amino acid sequence of the native Cry1Bb from position two (threonine) through at least the insecticidal core sequence of the toxin, the first two codons in the synthetic gene (at least with reference to SEQ ID NO:3 and its amino acid sequence translation at SEQ ID NO:4) encode for the incorporation of the amino acid residues methionine and alanine respectively at amino acid sequence positions one and two in the Cry1Bb proteins encoded by the nucleotide sequences intended for use in plants.

An insecticidal toxin protein expressed from the nucleotide sequences of the present invention comprises at least a core toxin fragment comprising and corresponding to approximately the first six-hundred and forty-three (643) amino acids of the native Cry1Bb protein as set forth in SEQ ID NO:2, or corresponding to approximately the first six-hundred forty four (644) amino acids of the Cry1Bb protein encoded by the synthetic nucleotide sequences of the present invention, as exemplified by the sequence as set forth in SEQ ID NO:4. However, a toxin protein produced from the nucleotide sequences of the present invention, which is substantially identical in amino acid sequence to a native Cry1Bb core toxin fragment, and which retains insecticidal activity to one or more lepidopteran pests previously demonstrated to be susceptible to at least the core toxin fragment, although consisting of an amino acid sequence slightly shorter than or slightly longer than the native core toxin but retaining no less insecticidal bioactivity than the native core toxin fragment, is also considered to be within the scope of the invention. SEQ ID NO:3, for example, comprises a synthetic nucleotide sequence which encodes an amino acid sequence variant of a Cry1Bb protein which retains lepidopteran insecticidal bioactivity equivalent to or greater than the bioactivity of the native Cry1Bb protein. SEQ ID NO:3 also encodes a core toxin fragment comprising from about amino acid position 1 through about amino acid position 644 as set forth in SEQ ID NO:4, corresponding substantially to a Cry1Bb core insecticidal crystal protein fragment, which retains bioactivity equivalent to or greater than that of the native Cry1Bb protein as set forth in SEQ ID NO:2. It is shown herein that a Cry1Bb fragment as set forth in SEQ ID NO:4 which corresponds to an amino acid sequence of from about 1 through about amino acid position 640 is sufficient to provide bioactivity equivalent to or greater than that of a native Cry1Bb protein. This would correspond to a native core toxin fragment of about the first six-hundred thirty nine (639) amino acids as set forth in SEQ ID NO:2. This would correspond to a native core toxin fragment of about the first six-hundred and thirty nine (639) amino acids as set forth in SEQ ID NO:2.

The overall amino acid sequence alignment of the native Cry1Bb to other known native Cry1 proteins provides insight into the relevant breakpoints between the sub-domains within the toxin fragment, and the relative breakpoint between the toxin domain and the protoxin domain of the native Cry1Bb full length protein. The native Cry1Bb amino acid sequence is comprised of (a) domain I from about amino acid one (1) through about amino acid two-hundred eighty-eight (288) as set forth in SEQ ID NO:2, corresponding to nucleotide position from about one (1) through about nucleotide position eight-hundred sixty-four (864) as set forth in SEQ ID NO:1; (b) domain II from about amino acid two-hundred eighty-nine (298) through about amino acid four-hundred ninety-six (496) as set forth in SEQ ID NO:2, corresponding to nucleotide position from about eight-hundred sixty-five (865) through about nucleotide position fourteen-hundred eighty-eight (1488) as set forth in SEQ ID NO:1; (c) domain III from about amino acid four-hundred ninety-seven (497) through about amino acid six-hundred forty-three (643) as set forth in SEQ ID NO:2, corresponding to nucleotide position from about fourteen-hundred eighty-nine (1489) through about nucleotide position nineteen-hundred twenty-nine (1929) as set forth in SEQ ID NO:1; and (d) the protoxin domain from about amino acid six-hundred forty-four (644) through about amino acid twelve-hundred twenty-nine (1229) as set forth in SEQ ID NO:2, corresponding to nucleotide position from about nineteen-hundred thirty (1930) through about nucleotide position thirty-six-hundred eighty-seven (3687) as set forth in SEQ ID NO:1.

The overall sequence of the amino acid variant Cry1Bb protein sequences disclosed herein resembles the native amino acid sequence, however the positions of the breakpoints for the sub-domains and the protoxin to toxin domain junction is shifted up one additional numerical value relative to the modification of the initiation sequences utilized for expression in planta, for example, as set forth in SEQ ID NO:4. The synthetic coding sequence is comprised of codons at nucleotide positions one through six (1-6) encoding an amino terminal MET-ALA di-peptide representing the first two amino acids in the amino acid sequence as set forth in SEQ ID NO:4, for example, engineered into the Cry1Bb sequence encoded by the synthetic sequences of the present invention. These two amino acid residues replace or are substituted for the native amino terminal LEU residue, therefore adding an additional amino acid residue at the amino terminus of the encoded Cry1Bb variant, resulting in the up-shift in position of the amino acid residues corresponding to the approximate breakpoints between the sub-domains I, II and III, and the toxin to protoxin domains.

Nucleotide sequences of the present invention which encode only an amino acid sequence corresponding to a Cry1Bb core toxin fragment are expected to be efficiently expressed in planta, however in some plants the core toxin fragment produced from expression from a nucleotide sequence which is less than full length when compared to the native Cry1Bb coding sequence may result in plants which exhibit physiological characteristics which are undesireable. In that event, it is likely that the construction of a nucleotide sequence encoding a Cry1 protoxin domain operatively linked to the coding sequence of the Cry1Bb core toxin fragment would stabilize the expression of the Cry1Bb protein. Therefore, fusion peptides of a Cry1Bb core toxin fragment to a protoxin domain of any other Cry1 toxin is contemplated as a specific embodiment of the invention. It is apparent that there can be some overlap between the nucleotide sequences encoding a Cry1Bb protein that is less than full length and the nucleotide sequences encoding the protoxin portions of Cry1 proteins.

The nucleotide sequences of the present invention, with reference to the sequence encoding the Cry1Bb or amino acid sequence variants of Cry1Bb are comprised of from about 50% to about 65% GC content, or from about 55 to about 64% GC content, or from about 60 to about 64% GC content, or about 64% GC content. One skilled in the art will recognize that this range of GC % is highly variable due to the redundancy of the genetic code, and so the GC % of a nucleotide sequence encoding a full length Cry1Bb or an insecticidal Cry1Bb amino acid sequence variant or insecticidal fragment thereof would range from about 46% or 48% GC on the low end up to about 60% or 65% GC or more depending upon the nature of the host cell in which expression is desired. This range is achieved without sacrificing substantially improved levels of expression in planta. The nucleotide sequences of the present invention correspond to sequences prepared by observing the amino acid sequence of the Cry1Bb native amino acid sequence and deducing the amino acid sequence intended for expression in planta. Substantially, the sequences of the present invention were prepared according to the methods as set forth in Brown et al. (U.S. Pat. No. 5,689,052) except that the starting material was not the native Cry1Bb coding sequence but was the native Cry1Bb amino acid sequence, and no partial sequences were prepared, but instead an entirely new nucleotide sequence was prepared using computer algorithms. The computer generated sequence was provided to a nucleotide synthesis service provider that completely synthesized the new sequence encoding the Cry1Bb amino acid sequence variants, confirmed the new sequence by sequencing the synthetic coding sequence in both directions, and provided the newly synthesized sequence in a cassette in a plasmid, the cassette flanked on either end by restriction endonuclease recognition sites engineered into the terminal ends of the synthetic sequence for the purpose of convenience in further manipulations designed for adding plant functional promoter sequences, plant functional intronic sequences, untranslated plant functional leader sequences, and plant functional 3′ transcription termination and polyadenylation sequences.

The DNA constructs of the present invention comprise fully synthetic structural coding sequences that enhance the performance of the sequence in plants. In a particular embodiment of the present invention, the enhancement method has been applied to design fully synthetic coding sequences encoding Cry1Bb variant insecticidal proteins. The structural genes of the present invention may optionally encode a fusion protein comprising an amino-terminal plastid or chloroplast transit peptide or a secretory signal sequence.

It should be apparent to one skilled in the art that the nucleotide sequences of the present invention can be constructed through several means. The nucleotide sequences of the present invention can be partially or even entirely constructed using a gene sequence synthesizer using, for example, phosphoramidite or related chemistries to link individual nucleotides into a polynucleotide sequence. Sequences which represent partial sequences encoding parts or fragments of the Cry1Bb or variant sequence can be inserted into the native sequence, or can be used as primers for linking the synthetic sequence to the native sequence so long as there is sufficient overlap or complementarity between all or a part of the synthetic sequence. The exemplified sequences can also be obtained or constructed by modifying the native gene encoding a Cry1Bb protein, for example, by point mutation or sequence replacement, and in particular using thermal amplification or other DNA synthesis and primer extension methodologies.

The nucleotide sequences of the present invention can also be used to form complete genes that encode proteins or peptides in a desired host cell. For example, those of skill in the art will recognize that the nucleotide sequences of the present invention can be illustrated in the sequence listing without termination codons in frame with and at the terminus of the coding sequence for the Cry1Bb protein. Nucleotide sequences encoding the Cry1Bb protein or variants thereof can be placed under the control of a promoter sequence for expression of the Cry1Bb protein in any host cell of interest. Methods and examples of these modifications are readily identifiable in the art.

The nucleotide sequences of the present invention can exist in either single or double stranded form. Double stranded forms are comprised of one strand that is complementary to the other strand and vice versa. The coding strand is referred to in the art as the strand or sequence containing the series of codons or base triplets that can be read as an open reading frame (ORF) to form a protein or peptide of interest. Expression of the protein necessarily involves transcription of the complementary or non-coding strand to produce a messenger RNA sequence which corresponds to the coding strand, which is used by the host cell's translational machinery as the template for the assembly of amino acids into a linear sequence corresponding to the sequence of the amino acid sequences of the present invention. Therefore, the subject invention includes the use of either the exemplified nucleotide sequences as set forth in SEQ ID NO:3, SEQ ID NO:5, SEQ ID NO:8, SEQ ID NO:11, and SEQ ID NO:13 and the corresponding complementary strands or sequences complementary to the exemplified nucleotide sequences. RNA molecules that are functionally equivalent to the exemplified nucleotide sequences are included in the subject invention.

It is specifically intended that the present invention includes equivalent and variants of the nucleotide sequences and amino acid sequences of the present invention, including but not limited to mutants, fusions, chimeras, truncations, fragments, and smaller or shorter genes and amino acid sequences. In particular, it is important to recognize that the intended sequences and variants thereof exhibit the same or similar characteristics relating to expression of toxins in plants, as compared to those specifically disclosed herein. As used herein, variants and equivalents includes reference to sequences which have nucleotide or amino acid substitutions, deletions whether internal and or terminal, additions, or insertions which do not materially affect the expression of the subject gene or genes or expression cassettes, and the resultant pesticidal activity in plants. Fragments that retain pesticidal activity are also included in this definition. Thus, nucleotide sequences that are smaller or shorter than those specifically exemplified are included in the subject invention, so long as the nucleotide sequence encodes a toxin that exhibits insecticidal bioactivity.

Genes and expression cassettes can be modified, and variations of these modifications can be readily constructed, using methods well known in the art. For example, methods for making individual nucleotide sequence changes described in the art as point mutations are well known in the art. In addition, commercially available nucleases are available for use in constructing sequences that are redacted in sequence in comparison to the nucleotide sequence that was used as the starting material. Such enzymes can be used to systematically excise various lengths of sequence from one end or the other of a linear nucleotide sequence.

In addition, restriction endonucleases can be used to construct fragments of sequences that can be moved into other sequences for construction of chimeras, variants, and modified sequences of the present invention.

It is apparent that equivalent genes will encode amino acid sequences corresponding to a Cry1Bb protein or variant thereof, and the protein will exhibit high amino acid sequence identity or homology with the native Cry1Bb protein or insecticidal amino acid sequence deletions, truncations, or variants thereof. The amino acid sequence homology will be the highest in the critical regions of the toxin that account for biological activity or are involved in the determination for three-dimensional configuration of the protein. For example, it is well known that the Cry1, Cry2, and Cry3 proteins fold into a three dimensional globular structure, and that each of the domains referred to hereinabove comprise each of the three globular domains which comprise the overall globular structure of these proteins. Particular folds, turns, or beta-sheet configurations require specific compositions of amino acid sequences to properly effectuate the overall intended insecticidal configuration and activity of the protein molecule. Incorporation of charged residues in regions in which there were previously no charged residues is likely to disrupt the configuration of the region, and likely therefore to disrupt the configuration of the overall protein, resulting in a loss of activity and the like. It is well known that each of the twenty naturally and most commonly occurring amino acids may be placed into various classes characterized as non-polar, uncharged-polar, basic, and acidic. Conservative substitutions, i.e., replacement of an amino acid of one class by an amino acid of the same type or class, fall within the scope of the subject invention so long as the substitution does not materially alter the exhibited biological activity of the Cry1Bb protein. Such conservative substitutions that are possible are well known in the art and can be readily identified using any biochemistry text book or equivalent resource. Nucleotide sequences encoding insecticidal fragments or even full length Cry1Bb proteins that hybridize to the nucleotide sequences as set forth herein under stringent conditions are believed to be within the scope of the present invention, in particular if the sequences are intended for use in expression of the Cry1Bb protein in plants. In particular, sequences that are from about 75% to about 80% identical in nucleotide sequence, or from about 80% to about 90% identical in nucleotide sequence, or from about 90% to about 99% identical in nucleotide sequence to the sequences of the present invention encoding Cry1Bb as set forth in SEQ ID NO:3, SEQ ID NO:5, SEQ ID NO:8, SEQ ID NO:11, and SEQ ID NO:13 are believed to be within the embodiments of the present invention.

In some cases non-conservative substitutions can be made which surprisingly increase the insecticidal activity, and do not reduce the in planta expression of the nucleotide sequence encoding the modified amino acid sequence variant Cry1Bb protein.

As used herein, reference to the word isolated nucleotide sequences and, or purified insecticidal toxin refers to these molecules when they are not associated with the other molecules with which they would be found in naturally occurring biological systems. For example, an isolated and/or purified nucleotide sequence encoding a Cry1Bb insecticidal protein or insecticidal fragment thereof would include its use in plants and in kits designed for use in detection of the molecules in biological samples. Such biological samples would include whole plants and or plant cells transformed to express a Cry1Bb protein or immunologically related Cry1Bb amino acid sequence variant, nucleotide sequences contained within said plants or plant cells, and extracts thereof; bacterial or fungal host cells which have been transformed to contain any of the nucleotide sequences of the present invention, including expression cassettes which are designed for use in plants and which are not intended for expression of a Cry1Bb or a Cry1Bb variant amino acid sequence in said bacterial or fungal host cells, and the like.

The expression cassettes and the coding sequences contained therein and the proteins expressed therefrom, i.e., the subjects of the present invention, can be introduced into a wide variety of microbial or plant hosts. In some embodiments of the present invention, transformed microbial hosts can be used in preliminary steps for preparing precursors, for example, that will eventually be used to transform, in preferred embodiments, plant cells and plants so that the plant and plant cells express the insecticidal Cry1Bb or variant proteins from the expression cassettes or coding sequences or substantial equivalents of the present invention. Bacillus, Salmonella, Clostridia, Escherichia, Yersinia, Pseudomonas, Pasteurella, Aeromonas, Agrobacterium, Rhizobacterium, and the like are representative genus' of bacteria which, when transformed with sequences of the present invention, are within the scope of the present invention, and methods are well known in the art for transforming and selecting recombinant microbes within the scope of the present invention.

In preferred embodiments, expression of the proteins of the present invention from the non-native nucleotide sequences of the present invention and from the expression cassettes of the present invention in plant cells, plant tissues, and plant hosts are within the scope of the invention. Methods for introducing heterologous nucleotide sequences into plant cells, plant genomes, plant chloroplasts and plastids and the like are well known in the art and include but are not limited to ballistic transformation methods, Agrobacterium or Rhizobacterium mediated transformation, vacuum mediated DNA uptake transformation methods, protoplast fusion methods, and the like are well known in the art and are within the scope of the present invention. These methods can be used for introducing a nucleotide sequence of the present invention into a plant cell, for example, into a crop plant such as corn, wheat, rice, oat, cotton, soybean, sunflower, cauliflower, broccoli, canola or rape seed, and the like. In addition, fruit trees such as apples, pears, peaches, apricot, orange, lemon, lime, grapefruit, and the like, and vines such as grapes, and berries such as blueberries and strawberries, potato, sugar cane, beans and the like, and grasses such as bluegrass, brome, crabgrass, creeping bentgrass, fescue, ryegrass, Saint Augustine, timothy, zoysia, and the like and forage plants such as alfalfa, and clover, and the like, are within the scope of the present invention. The nucleotide sequences encoding Cry1Bb and amino acid sequence variants and the expression cassettes of the present invention are particularly well suited as exemplified herein for providing high-level expression of the Cry1Bb insecticidal proteins, insecticidal fragments, and insecticidal variants thereof in planta.

Agronomically and commercially important products and/or compositions of matter including but not limited to animal feed, commodities, and corn products and by-products that are intended for use as food for human consumption or for use in compositions that are intended for human consumption including but not limited to corn flour, corn meal, corn syrup, corn oil, corn starch, popcorn, corn cakes, cereals containing corn and corn by-products, and the like, and transgenic Cry1Bb broccoli, transgenic Cry1Bb cauliflower, transgenic Cry1Bb squash, transgenic Cry1Bb melons, transgenic Cry1Bb cucurbits, transgenic Cry1Bb soybean, transgenic Cry1Bb canola, transgenic Cry1Bb wheat, transgenic Cry1Bb tomatoes, transgenic Cry1Bb fruit trees, and the like are intended to be within the cope of the present invention if these products and compositions of matter contain detectable amounts of the nucleotide sequences or Cry1Bb proteins set forth herein.

As set forth in the examples below, the inventors herein demonstrate that a synthetic nucleotide sequence encoding an insecticidal variant amino acid sequence substantially equivalent to the native Cry1Bb1 insecticidal protein exhibits high levels of expression in plants, in particular when the nucleotide sequence is embedded within a larger nucleotide sequence designed for expression of a coding sequence such as the synthetic sequence when present in plant cells. Therefore, the expression cassette, and the nucleotide sequence encoding the Cry1Bb protein, are excellent insect resistant management tools, in particular when combined with other Bt or other types of insect toxin proteins co-expressed along with the Cry1Bb protein or when combined with topically applied insecticidal chemical agents, each exerting their specific insecticidal activity upon a target insect by means of a different mode of action than that exhibited by the Cry1Bb protein.

The inventors herein set forth examples of how these insecticidal agents work, in particular by using Cry1A type resistant Diamondback Moth and Cry1A type resistant European Corn Borer. Larvae exposed to Cry1A proteins exhibit virtually no level of inhibition. However, exposure of these Cry1A resistant larvae to Cry1Bb protein results in mortality, indicating that the Cry1B protein functions to cause insecticidal effects for these species in a way that is different from the means used by the Cry1A toxins. The inventors therefore demonstrate the utility of the protein as a resistance management tool, and demonstrate the improvement in levels of expression of the Cry1Bb protein in plants from the unique and novel expression cassettes disclosed herein.

### 5.0 EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### In Vitro Bioactivity of Cry1Bb against Dipel™ Resistant European Corn Borer

Lepidopteran species that develop resistance to insecticidal proteins derived from Bacillus thuringiensis or Bt) bacteria tend to do so through multiple, unexpectedly dominant alleles. The development of resistance to insecticidal proteins under laboratory conditions appears to be more complex and more difficult to control than many experts have assumed and could be of importance to regulatory officials responsible for monitoring crops that are engineered to produce such proteins. It is possible that target plant pests could develop resistance in the wild to biological pesticidal agents such as B. thuringiensis crystal toxin proteins. An extensive review of the literature in this area can be found in Ferre et al. (Annu. Rev. Entomol. 2002,47:501-533).

Recombinant plants that express Cry1A B. thuringiensis crystal protein toxins have been commercialized since 1996. Requirements for resistance management strategies have been implemented in order to decrease the likelihood of the development of resistance. Statistical studies indicate that pest resistance to the Cry1A class of proteins is likely to develop without the implementation of resistance management strategies, and even then, likely to develop if the Cry1A plants are maintained in the fields in the absence of an additional insecticidal agent exhibiting a mode of action different from the mode of action of the Cry1A protein toxin. It has been demonstrated with chemical insecticides and with antibiotic selection that resistance is less likely to develop when agents exhibiting different modes of action are used in combination and directed to a common insect pest species. Cry1A resistant strains of lepidopteran larvae have been developed under tightly controlled laboratory conditions. In particular, a Cry1A-type diamondback moth race has been identified which is insensitive to high levels of Cry1A toxin. It is logical to assume that a pest sensitive to both Cry1A and Cry1B type toxins would be insensitive to Cry1B type toxins if the pest develops resistance to Cry1A type toxins. This assumption is based primarily on the degree of relationship of Cry1A to Cry1B proteins. These proteins belong to the same Cry1 class of B. thuringiensis δ-endotoxin proteins, and are ontologically related. However, Donovan et al. (WO95/04146) demonstrated that diamondback moth strains resistant to Cry1A-type B. thuringiensis δ-endotoxins retain sensitivity to Cry1Bb, highlighting the utility of this protein as a resistance management tool. In the absence of resistance management strategies employing two or more modes of action, Bt toxin levels in compositions used for on planta (topical) application or for in planta expression should be maintained at high levels in order to prevent or significantly delay the onset of resistance. Alternatively, combining Bt toxins exhibiting different modes of action, i.e., each toxin being toxic to the same insect species but each toxin exerting it's effect by a means different from that of the other toxin, would also be a means for preventing the onset of resistance.

Donovan et al. demonstrated bioactivity of Cry1Bb1 in in vitro bioassays against a number of lepidopteran species. In particular, bioactivity was demonstrated against gypsy moth (Lymantria dispar), European corn borer (Ostrinia nubilalis), fall army worm (Spodoptera frugiperda), soybean looper (Pseudoplusia includeizs), diamondback moth (Plutella xylostella), and cabbage looper (Trichoplusia ni).

The inventors herein demonstrate that a synthetic sequence encoding a Cry1Bb insecticidal protein toxin exhibits high levels of expression in plants, and is therefore an excellent insect resistance management tool, in particular when combined with other Bt or other types of insect toxin proteins or chemical agents, each exerting their specific insecticidal activity upon a target insect by means of a different mode of action than that exhibited by Cry1Bb.

Cry1Bb bioactivity against a variety of lepidopteran insects such as European corn borer (ECB, Ostrinia nubilalis)) and fall army worm (FAW, Spodoptera frugiperda) has previously been demonstrated (Donovan et al., U.S. Pat. Nos. 5,679,343 & 5,616,319). Diamondback moth strains resistant to Cry1A-type B. thuringiensis δ-endotoxins retain sensitivity to Cry1Bb, highlighting the utility of this protein as a resistance management tool (Donovan et al., supra). ECB is presently controlled on a significant portion of the planted transgenic maize acreage by expression of Cry1A-type B. thuringiensis δ-endotoxins. This presents an opportunity for the development of ECB populations resistant to Cry1A-type B. thuringiensis δ-endotoxins.

A population of ECB selected in the laboratory for resistance to DIPEL™, a commercially available mixture of Bacillus thuringeisis spores comprising Cry1A-type and Cry2A-type endotoxins, was tested for sensitivity to Cry1Bb to determine if Cry1Bb could control Cry1A-type resistant ECB (Huang et al., Science 284:965-967; 1999). The test was conducted by exposing larvae to solubilized B.t. δ-endotoxin incorporated into an artificial diet. Typical levels of Cry1Ab that are attained in commercially available transgenic maize ranges from about 10 to about 20 ppm. The results are shown in Table 1. Cry1Ab resistant ECB were insensitive to levels of Cry1Ab which have not been attained in commercially available transgenic plants. However, these same Cry1Ab resistant ECB retained sensitivity to Cry1Bb at levels routinely attained in transgenic plants as described herein below. These results suggest that Cry1A resistant ECB, and presumably other lepidopteran larvae, which develop resistance to Cry1A type δ-endotoxins should exhibit sensitivity to Cry1Bb.

TABLE 1
ECB sensitivity to Cry1Bb

| | Dipel ™ Resistant ECB | Dipel ™ Sensitive ECB |
|---|---|---|
| Endotoxin | (LC50 in ppm) | (LC50 in ppm) |
| Cry1Ab | >50 ppm | 0.08-0.4 ppm |
| Cry1Bb | 0.32-1.6 ppm | <0.32 ppm |

### Example 2

### Construction of Synthetic Nucleotide Sequences Encoding Cry1Bb

Coding sequences derived from Bacillus thuringiensis do not express well, if at all, in plants, in general because plant nucleic acid sequences tend to exhibit from about 50% to about 60% or greater GC content, while nucleic acid sequences derived from Bacillus thuringiensis tend to exhibit from about 60 to about 70% AT content. Generally, it has been demonstrated that reduction of AT rich sequences in BT protein encoding regions intended for expression in plants results in improvements in in planta levels of expression of the coding region. One means for decreasing the level of AT composition in Bt coding sequences comprises obtaining the amino acid sequence of a Bt protein and constructing a gene for expression in plant cells by using where possible a codon for each particular amino acid in the protein sequence which reduces the overall composition of AT in the coding sequence such that the overall GC content of the coding sequence tends to be from about 50% to about 60% or greater, and which results in a coding sequence which is substantially devoid of regions containing stretches of A or T or A and T of less than five or six nucleotides in length. Examples of non-native nucleotide sequences for use in in planta expression of Cry1Bb and Cry1Bb amino acid sequence variants, analogs, and homologs are illustrated at SEQ ID NO:3, SEQ ID NO:5, SEQ ID NO:8, SEQ ID NO:11, and SEQ ID NO:13, the designated Cry1Bb open reading frames of which correspond to amino acid sequences comprising a Cry1Bb insecticidal protein or insecticidal fragment thereof as set forth in SEQ ID NO:4, SEQ ID NO:7, SEQ ID NO:10, SEQ ID NO:12, and SEQ ID NO:14.

The nucleotide composition of each of the coding sequences intended for improved expression of Cry1Bb toxins or insecticidal fragments thereof as set forth in SEQ ID NO:3, SEQ ID NO:5, SEQ ID NO:8, SEQ ID NO:11, and SEQ ID NO:13 are comprised of between 55 and 65% GC. These non-native and synthetic sequences encoding Cry1Bb amino acid sequences and Cry1Bb amino acid sequence variants were constructed according to the method of Brown et al. substantially as set forth in U.S. Pat. No. 5,689,052, except that the resulting nucleotide sequence was not partially obtained from starting material originating from native B. thuringiensis nucleotide sequences. Instead, the complete synthetic Cry1Bb coding sequence was prepared by nucleotide synthesis service providers after providing one or more nucleotide synthesis service providers with all or a part of the desired terminal or resulting nucleotide sequence for encoding Cry1Bb in plants. The resulting sequences comprise pre-selected nucleotide sequences encoding at least an insecticidal portion or fragment of a Cry1Bb, or a Cry1Bb amino acid sequence variant, wherein the pre-selected nucleotide sequence is adjusted relative to the native nucleotide sequence to be more efficiently expressed in plants in comparison to the levels of expression of the native nucleotide sequence encoding a Cry1Bb insecticidal protein. While the nucleotide sequences disclosed herein are but a few examples of Cry1Bb coding sequences which are shown herein to function in plants to produce insect inhibitory effective amounts of Cry1Bb in plant cells and in plant tissues, it should be understood that there are multiples of other sequences which may work as well to allow for expression of Cry1Bb in plants, keeping in mind the limitations on codon usage and specific nucleotide composition described herein above. These sequences can be linked to plant functional promoters and 3′ end transcription termination and polyadenylation sequences, as well as other types of expression modulating elements for optimizing the expression of each sequence in a desired genus, species, or variety of plant cell or plant tissue. It is believed that a nucleotide sequence encoding all or an insecticidal fragment of a Cry1Bb or a Cry1Bb amino acid sequence variant, or the like, which is identical to or approximately between 95-99% identical to the sequences set forth herein would function as well as those sequences described herein for expression of said protein or proteins in plants, and are specifically intended to be within the scope of the present invention.

### Example 3

### Cassettes Encoding Cry1Bb and Variants for Use in Plants

A variety of genetic elements were combined together with Cry1Bb coding sequences in plant transient expression and transformation vectors in order to identify sequences comprising plant expression cassettes likely to provide commercially useful levels of expression of Cry1Bb protein in plants. The individual elements selected for use herein are exemplary only, and in the examples herein, the elements selected were chosen particularly because the exemplary plants tested herein are maize plants and the selected elements have been previously shown to function in maize plants as promoters, intronic sequences, plastid targeting sequences, leader sequences, and termination sequences. Various promoters, 5′ untranslated leaders, intron sequences, plastid targeting sequences, and 3′ end transcription termination and polyadenylation sequences were grouped together in operable combinations with synthetic Cry1Bb coding sequences. Promoters were selected from the CAMV-e35S(P-CaMV.e35S) promoter and the figwort mosaic virus (P-FMV.35S) promoter, however, the skilled artisan will recognize that many other plant functional promoters known in the art will suffice in place of the two selected for exemplary purposes. Other elements are to be construed as being exemplary as well. Untranslated leader sequences were selected from the wheat chlorophyll a/b binding protein leader (L-Ta.Cab), and the rice beta tubulin leader (L-Os.βTub). Intronic sequences were selected from the rice actin 1 gene intron (1-Os.Act1) and the rice phenylalanine ammonia lyase gene intron (1-Os.PAL). A nucleotide sequence encoding a Zea mays ribulose bis-phosphate carboxylase small subunit plastid targeting sequence was used in some vector constructions (TS-Zm.rbcs) (Lebrun et al., 1987, NAR 15:4360). The nucleotide sequence encoding the Zea mays plastid targeting peptide is set forth herein at least from nucleotide position 2644 through nucleotide position 3040 of SEQ ID NO:8, and consists of a maize genomic coding fragment containing an intron sequence (nucleotide 2791 through nucleotide 2953 of SEQ ID NO:8) as well as a sequence encoding a duplicated proteolytic cleavage site present in the resulting plastid targeting peptide amino acid sequence (first of said sequences encoding said duplicated cleavage sites being positioned from nucleotide positions 2644 through 2790 and further, after excision of the intron, including the nucleotides at position 2954 through 3040, and the second of said sequences encoding said duplicated cleavage sites being positioned within the amino acid sequence encoded by nucleotides 2954 through 3040 of SEQ ID NO:8, and derived from plastid targeting sequence zmS 1; Russell et al., 1993). Direct translational fusions of the TS-Zm.rbcs to the amino terminus of the preferred sequences encoding insecticidal proteins herein are useful in obtaining elevated levels of the insecticidal protein in transgenic maize.

In-frame fusions of the TS-Zm.rbcs nucleic acid sequence (as set forth at nucleotides 2644 through 3040 of SEQ ID NO:8) to the gene sequence encoding a Cry1Bb protein (SEQ ID NO:3) can be effected by ligation of the NcoI site at the 3′ (C-terminal encoding) end of the TS-Zm.rbcs coding sequence with the 5′ NcoI site (N-terminal encoding) of the Cry1Bb coding sequence. The use of plastid targeting sequences linked to a Cry1A or a Cry2Ab insecticidal toxin protein has been demonstrated to be effective in improving the level of protein accumulation in a plant cell. However, it is not known which Bt proteins can benefit from the function of a linked plastid targeting peptide (see Corbin et al., WO 00/26371). Transcription termination and polyadenylation sequences were selected from the wheat Hsp17 gene termination sequence (T-Ta.Hsp17) and the rice lactate dehydrogenase gene termination sequence (T-Os.Ldh), and are identified as features by sequence location within cassette sequences provided herein.

In order to effectively monitor levels of expression of Cry1Bb in transient expression systems and in transgenic plants, immunological assays were developed using antibodies specific for binding to Cry1Bb protein. Antibodies to purified Cry1Bb protein were produced by means well known in the art. Quantitative ELISA assays were developed for measuring Cry1Bb protein levels in various assays and compositions of matter. A Cry1Bb pure protein crystal slurry was obtained from Bacillus thuringiensis strain EG7283 (NRRL B-21111, Donovan et al., U.S. Pat. No. 5,679,343). The crystals were solubilized, and the protein quantified and sent to a service provider for polyclonal antisera generation (Celsis Laboratory, St. Louis). Rabbits were immunized with the antigen according to standard immunization procedures, resulting in a high titer Cry1Bb antisera.

IgG was purified from the rabbit sera and used as a capture antibody in a sandwich ELISA. The ELISA assay was performed by first coating a 96-well polystyrene ELISA plate (Nunc, Denmark) with a high titer polyclonal anti-Cry1Bb capture antibody at a concentration of 125 ng IgG/well. The plate was allowed to incubate overnight at 4° C. in a sealed, humid container. The following day, the plate was washed and samples were loaded beside a standard curve comprising purified Cry1Bb protein. Appropriate buffer blanks and positive/negative controls were included. The Cry1Bb test samples, standards and controls were incubated overnight at 4° C. with the bound capture antibody and a horseradish peroxidase-conjugated secondary antibody. The following day, plates were washed and treated with a TMB substrate solution to allow for a colorimetric detection. Concentrations of Cry1Bb were determined in each sample by extrapolating an optical density reading against a Cry1Bb standard curve. Results are reported on parts per million, fresh weight basis.

Four distinct expression cassettes were tested in transient corn protoplast expression assays and evaluated for expression by quantitative ELISA and efficacy against ECB in diet overlay bioassay. The vectors and elements tested are outlined in Table 2.

TABLE 2
Composition of Corn Protoplast Cry1Bb Transient Expression Vectors and Expression Cassettes

| | | | | | | | | | | | | |
|---|---|---|---|---|---|---|---|---|---|---|---|---|
| 33731^{a} | P-FMV | : | L-Os.βTub | : | I-Os.PAL | : | | | cry1Bb1 | : | T-Os.Ldh | SEQ ID NO:5 |
| 40227^{a} | P-CaMV.e35S | : | L-Ta.Cab | : | I-Os.Act1 | : | | | cry1Bb1 | : | T-Ta.Hsp17 | SEQ ID NO:11 |
| 33732^{a} | P-FMV | : | L-Os.βTub | : | I-Os.PAL | : | TS-Zm.rbcs | : | cry1Bb1 | : | T-Os.Ldh | SEQ ID NO:8 |
| 40228^{a} | P-CaMV.e35S | : | L-Ta.Cab | : | I-Os.Act1 | : | TS-Zm.rbcs | : | cry1Bb1 | : | T-Os.Ldh | SEQ ID NO:13 |

(:) represents separation of various amorphous nucleotides between functional genetic elements; P indicates promoter element; L indicates untranslated 5 leader sequence; I indicates intron sequence; TS indicates transit peptide (containing an embedded intron in this example); T indicates plant functional transcription termination and polyadenylation sequence; SEQ ID NO: indicates the particular sequence listing number exemplifying the indicated composition and expression cassette;
(a) designates pMON plasmid number corresponding to the operably linked genetic elements on same line. Each expression cassette contains a sequence encoding an identical Cry1Bb variant amino acid sequence; pMON33731 expression cassette was transferred into a plant transformation vector to create pMON33733. PMON33732 expression cassette was transferred into a plant transformation vector to create pMON33734.

Expression from the indicated vectors and insecticidal bioactivity of the transient protoplasts was tested in a maize transient expression assay. Cry1Bb protein expression was measured by ELISA as described above, and insecticidal activity was measured by feeding transient maize protoplasts to ECB larvae. The results obtained are shown in Table 3.

TABLE 3
Cry1Bb corn protoplast expression and efficacy against ECB larvae.

| Vector | ELISA | |
|---|---|---|
| pMON: | (ppm) | Mortality |
| 33731 | 0.21 | 0.92 |
| 40227 | 0.34 | 0.92 |
| 33732 | 0.05 | 0.5 |
| 40228 | 0.1 | 0.83 |
| no DNA | 0 | 0.17 |

Vectors encoding Cry1Bb protein not targeted for chloroplast uptake expressed greater levels of Cry1Bb protein than vectors encoding plastid-targeted Cry1Bb fusion proteins. However, Cry1Bb protein expressed from either form of expression cassette resulted in effective levels of mortality in comparison to the negative control, but non-targeted expression was better likely due to the elevated levels of Cry1Bb protein accumulation. In any event, it is nonetheless clear that either form of expression cassette would be equally efficacious in delivering Cry1Bb-mediated insect control in transgenic plants.

### Example 4

### Plant Transformation and Expression

Transgenic corn plants expressing Cry1Bb protein were produced after transformation with plant transformation vectors containing substantially the same expression cassettes exemplified in the plasmids as set forth in Table 2. Expression of the Cry1Bb protein produced in these transgenic corn plant events was compared and was observed to be significantly higher in plants produced after transformation with vectors containing expression cassettes in which the Cry1Bb protein or variant was targeted to the chloroplast. pMON33733 contains an expression cassette as set forth in SEQ ID NO:5 comprising a sequence containing an FMV35S promoter (P-FMV), a rice beta tubulin untranslated leader sequence (L-Os.ptub), a rice phenylalanine ammonia lyase intron sequence (1-Os.PAL), a synthetic Cry1Bb variant coding sequence (cry1Bbl), and a rice lactate dehydrogenase transcription termination and polyadenylation sequence (T-Os.Ldh). pMON33734 contains an expression cassette as set forth in SEQ ID NO:8 consisting of a sequence containing a FMV35S promoter (P-FMV), a rice beta tubulin untranslated leader sequence (L-Os.ptub), a rice phenylalanine ammonia lyase intron sequence (I-Os.PAL), a sequence encoding a maize ribulose bis-phosphate carboxylase small subunit chloroplast transit peptide (CTP or TP-Zm.rbcs) fused in-frame to a synthetic Cry1Bb variant coding sequence (cry1Bb1 variant), and a rice lactate dehydrogenase transcription termination and polyadenylation sequence (T-Os.Ldh). Both vectors also contain a cassette consisting of a CaMV35S promoter sequence, a neomycin phosphotransferase (NPI) coding sequence, and a nopaline synthase transcription termination and polyadenylation sequence that confers paromomycin resistance to transformed plant tissue and is used as a selectable marker. One skilled in the art will recognize that any element that can be used as a selectable marker can function in place of the present nptII gene. For example luc, bar, phnO, glyphosate tolerant epsps alleles, gox, and the like, can be used along with or in place of nptII as a selectable marker for identifying plant cells and plants that have been transformed to contain a gene of interest such as a synthetic sequence encoding an insecticidal protein. Transgenic corn plants resistant to paromomycin were derived essentially as described in U.S. Pat. No. 5,424,412. Leaf discs from Ro plants were placed in wells with ECB larvae and scored for ECB resistance to identify plants expressing toxic or insect inhibitory levels of Cry1Bb protein. Ninety-six (96) independent events were obtained after transformation with pMON33733 and selection in the presence of paromomycin. Twelve (12) of these were identified by leaf disc feeding bioassay to exhibit resistance to European corn borer, and six (6) of these ECB resistant plants exhibited strong s resistance. Ninety-four (94) independent events were obtained after transformation with pMON33734 and selection in the presence of paromomycin. Plants in this group exhibited from about one (1) ppm to about one-hundred sixty (160) ppm of Cry1Bb protein as measured by ELISA. Eighteen (18) of these were identified by leaf disc feeding bioassay to exhibit resistance to ECB, and eleven (11) of these exhibited strong resistance. Plants in this group exhibited from about one (1) ppm to about three-hundred forty five (345) ppm of Cry1Bb protein as measured by ELISA.

Leaf tissue from ECB resistant, independently transformed transgenic events in the R₀ stage was subjected to quantitative analysis of Cry1Bb protein levels by the quantitative ELISA assay. Tissue samples from fresh R₀ corn leaf discs were sampled from each plant directly into a 1.5 mL Sarstedt microcentrifuge tube. Plants were sampled at about the V3 leaf stage. Each leaf sample was weighed and TBA buffer (100 mM Trizma Base, pH 7.5; 100 mM sodium borate; 0.2% (w/v) L-ascorbic acid (added immediately before use); 0.05% Tween-20; 5 mM MgCl₂ (6H₂O)) was added at a 1:100 tissue to buffer ratio. The leaf tissue was homogenized into the buffer with a Wheaton overhead stirrer for ˜20 seconds. The homogenized leaf tissue was then subjected to about 12,000 g for 5 minutes in a microcentrifuge, separating the plant tissue solids from the solubilized protein supernatant. This extract supernatant was added to wells in microtiter plates and subjected to analysis by D ELISA.

Protein blot analysis confirmed that the increased level of cross-reactive material produced by pMON33734 events was due to increased accumulation of an approximately 66 kDa protein that co-migrates with a 66 kDa protein which accumulates in pMON33733 events and which is immuno-reactive with anti-Cry1Bb antiserum. The 66 kDa protein is consistent in mass with the predicted size of the Cry1Bb toxin domain and may be derived by proteolysis of the about 130,000 kDa full length Cry1Bb variant protein protoxin after expression in planta. The native Cry1Bb full length protein produced from Bacillus thuringiensis strain EG5847 can be proteolytically cleaved to release an insecticidal protein which is approximately 66 kDa, corresponding to the core toxin domain of Cry1Bb, which likely is represented by the amino acid sequence from about position one (1) through about position six-hundred forty three (643) as set forth in SEQ ID NO:2. The data reported herein suggests that the targeting peptide fused to the N-terminus of the Cry1Bb protein and expressed in events transformed with pMON33734 was efficiently processed or removed, and therefore that the insecticidal protein toxin must be localized within the chloroplast.

To establish that events produced from transformation with the plastid targeted Cry1Bb expression vector pMON33734 resulted in localization of the toxin protein to the chloroplast, samples of these plants were subjected to protein immuno-gold labeling and electron microscopy and compared to samples from events transformed with the expression vector pMON33733. Immuno-gold labeling showed the presence of gold particles and thus Cry1Bb protein only in the chloroplasts within the cells derived from events produced by transformation with pMON33734, indicating that the protein was properly targeted using the CTP sequence. In contrast, Cry1Bb protein was found throughout the cells derived from events produced by transformation with pMON33733. Gold labeling of cells in an isogenic control line, H99, was not apparent.

Events derived from transformation with the pMON33734 vector produced a higher percentage of events exhibiting ECB tolerance. Leaf disks from Ro plants were exposed to neonate ECB larvae and scored for feeding damage as previously described (Armstrong et al, 1995, Crop Science 35:550-557). While non-transgenic control disks were totally consumed, disks from transgenic lines exhibiting resistance to ECB feeding were readily identified. The percentage of events-exhibiting any ECB resistance was markedly increased in events transformed with the vector pMON33734 (Table 4). Twice as many events with strong ECB resistance were obtained when pMON33734 was used relative to events selected after transformation with the vector pMON33733. Thus, transformation of plant cells using the vector encoding the chloroplast targeted Cry1Bb surprisingly increases the probability of obtaining a transgenic line exhibiting insecticidal properties, insect toxicity, and ECB resistance.

TABLE 4
Expression of Cry1Bb in R₀ maize

| | | | Total | | | | | | |
|---|---|---|---|---|---|---|---|---|---|
| | Total | Total | Strong | 0-10 | 10-50 | 50-150 | 150-200 | >200 | Highest |
| Vector | Events¹ | ECB R² | ECB R³ | ppm⁴ | ppm | ppm | ppm | ppm | ppm |
| pMON33733 | 96 | 12 | 6 | 3 | 6 | 2 | 1 | 0 | 160 |
| (non- | | (12.5%) | (6.3%) | | | | | | |
| targeted) | | | | | | | | | |
| pMON33734 | 94 | 18 | 11 | 5 | 3 | 6 | 2 | 2 | 345 |
| (plastid | | (19%) | (12%) | | | | | | |
| targeted) | | | | | | | | | |

¹Number of paromomycin resistant plant events obtained
²Number and percentage of the total (in parenthesis) plants exhibiting ECB resistance
³Number and percentage of the total (in parenthesis) plants exhibiting strong ECB resistance.
⁴parts per million (or ug/gm fresh weight tissue) of Cry1Bb as determined by ELISA.

### Example 5

### Herbicide Resistant Transgenic Maize Expressing Cry1Bb

The expression cassette in pMON33732, identical to the expression cassette in pMON33734, as set forth in SEQ ID NO:8, demonstrated insect inhibitory effective levels of Cry1Bb expression in transgenic maize. This expression cassette was subsequently engineered into two alternative monocotyledonous plant transformation vectors that contain an identical gene expression cassette permitting recovery of transgenic maize plants with glyphosate tolerance. The gene expression cassette conferring glyphosate tolerance consists of a previously described rice actin Act1 promoter and intron sequence, an Arabidopsis thaliana EPSPS untranslated leader sequence, a sequence encoding an Arabidopsis thaliana plastid targeting peptide, a sequence encoding a glyphosate insensitive EPSPS (enol pyruvyl shikimate 3 phosphate synthase) or AroA protein referred to herein and in the literature as CP4, and a NOS 3′ transcription termination and polyadenylation sequence. pMON33750 is a composite vector containing two expression cassettes. The cassette expressing Cry1Bb is identical to the cassette present in pMON33734. The other cassette encodes a EPSPS enzyme which confers tolerance to glyphosate herbicide as the selectable marker in place of the NptII coding sequence in pMON33734. pMON33750 was digested with MluI restriction endonuclease to release a DNA fragment containing only the Cry1Bb and glyphosate tolerance expression cassettes, which was purified and used to transform maize cells using ballistic methods, followed by glyphosate selection, using methods well known in the art. Another composite vector containing both the Cry1Bb and glyphosate tolerance cassettes, pMON40213, was used to transform maize cells using Agrobacterium-mediated transformation, by methods well known in the art. Maize cells transformed with DNA from pMON33750 or with pMON40213 were subsequently regenerated into glyphosate tolerant plants and screened for expression of Cry1Bb protein using the ECB leaf disk feeding bioassay and Cry1Bb quantitative ELISA (Armstrong et al., supra.).

Transgenic pMON33750 and pMON40213 S2 (homozygous, self pollinated) progeny maize plants were subsequently assayed for expression of Cry1Bb protein. Expression of Cry1Bb protein was detectable at all stages of development assayed, with the highest levels detected at the V12 stage of development. This data confirmed that the pMON33750 and pMON40213 transgenes remain active after multiple generations and throughout plant development, two critical characteristics for agronomically useful transgene-mediated insect control (Table 5). High level insecticidal transgene expression at later stages of plant development is especially useful in providing season long control of insect pests.

TABLE 5
Expression of Cry1Bb in Maize at V4, V8 and V12 leaf stages

| | | V4² | V8² | V12² |
|---|---|---|---|---|
| | Event¹ | (Cry1Bb, ppm) | (Cry1Bb, ppm) | (Cry1Bb, ppm) |
| | pMON33750 | | | |
| 1 | RAB138 | 5 | 3 | 26 |
| 2 | RAB150 | 7 | 11 | 45 |
| 3 | RAB152 | 7 | 8 | 46 |
| 4 | RAB158 | 5 | 9 | 36 |
| 5 | RAB167 | 10 | 9 | 54 |
| 6 | RAB169 | 11 | 8 | 56 |
| 7 | RAB175 | 18 | 9 | 38 |
| 8 | RAB183 | 15 | 9 | 64 |
| 9 | RAB174 | 16 | 8 | 20 |
| 10 | RAB180 | 12 | 9 | 22 |
| 11 | RAB188 | 10 | 14 | 56 |
| 12 | RAB201 | 13 | 15 | 44 |
| 13 | RAB210 | 12 | 9 | 52 |
| 14 | RAB226 | 11 | 11 | 55 |
| 15 | RAB249 | 10 | 9 | 43 |
| 16 | RAB252 | 12 | 16 | 72 |
| | pMON40213 | | | |
| 1 | RAA376 | 8 | 9 | 55 |
| 2 | RAA401 | 5 | 9 | 49 |
| | LH198 | 0 | 0 | 0 |

1-individual events in this column were selected after transformation with nucleotide sequences present in the 5 plasmid indicated in boldface type
2-events were sampled at either the 4, 8, or 12 leaf stage and the level of Cry1Bb protein was determined using ELISA as described herein, and reported as parts per million of total protein

In order to compare levels of ECB control by Bt insecticidal transgenic maize, three pMON22750 transgenic maize events were grown in field conditions and compared to a commercially available transgenic maize line, MON810 (Monsanto Company, St. Louis, Missouri) expressing a Cry1A B. thuringiensis insecticidal crystal protein toxin. First and second generation European Corn Borer broods (ECB 1 and ECB2, respectively) were evaluated and the results are shown in Table 6. In this experiment, the non-transgenic control sustained extensive damage while the transgenic maize expressing either a plastid targeted Cry1Bb (RAB172, 401, and 150) or Cry1A (MON810) both displayed excellent control of ECB1 and ECB2. Control of ECB infestation and feeding damage by plants expressing Cry1Bb protein was statistically indistinguishable from control of ECB infestation and deeding damage by plants expressing Cry1A protein.

The stand-alone ECB control exhibited by maize expressing Cry1Bb thus satisfies the key redundant control requirement for an insect resistance management strategy that would be based on a two gene product. This data and aforementioned diet bioassay data demonstrating activity of Cry1Bb against insects that are resistant to Cry1A-type B. thuringiensis δ-endotoxins indicates that maize expressing the Cry1Bb insecticidal protein could be used to combat infestations of Cry1A-type resistant European corn borer populations. Infestations of Cry1A-type resistant insects could be controlled either by exclusive use of plants expressing Cry1Bb or by genetically combining the Cry1Bb transgene with at least one additional insecticidal transgene in a single plant (Corbin et al., WO00/26371). Examples of the second transgene include cry1Aa, cry1Ab, cry1Ac, cry1F, cry2Ab, and various hybrid genes formed from cry1A and cry1F coding sequences expressing chimeras exhibiting the same or improved insecticidal bioactivity of the native proteins from which the hybrids were formed. All transgenic events expressing an insecticidal Cry1 protein exhibited significantly better insect resistance than the control (p<0.05).

TABLE 6
Performance of Transgeuic Maize in field conditions.

| Cry | | ECB1^{A} | | ECB2 | |
|---|---|---|---|---|---|
| Gene | Event | 0-9 leaf | SE^{B} | cm tunnel | SE^{C} |
| 1Bb | RAB172 | 0.55 | 0.63 | 0.43 | 1.01 |
| 1Bb | RAB401 | 0.20 | 0.52 | 0.00 | 0.83 |
| 1Bb | RAB150 | 0.07 | 0.52 | 0.14 | 0.83 |
| 1A | MON810 | 0.25 | 0.45 | 0.32 | 0.72 |
| Control | non-transgenic | 8.90 | 0.45 | 25.08 | 0.72 |

^{A}:leaf damage rating scale of 0-9 where 0 represents no damage/ excellent control and a 9 represents extreme damage/ no control.
^{B}:SE indicates standard error or standard deviation from the indicated leaf damage rating
^{C}:SE indicates the standard error or standard deviation from the indicated tunneling distance in centimeters

### Example 6

### Maize Expressing Cry1Bb Exhibits Improved Fall Army Worm Control

Although ECB is the primary maize insect pest in North America, other insects such as the fall armyworm (FAW or Spodoptera frugiperda) can also cause significant economic loss, particularly in South America. pMON33750 transformed maize events were challenged with FAW larvae to determine if transgenic maize expressing Cry1Bb could provide improved control of insects other than ECB. The results are shown in Table 7. Several events expressing Cry1Bb demonstrated excellent protection against heavy natural FAW infestation in field tests. In at least one event (RAB172), FAW control was statistically indistinguishable from control conferred by plants expressing only Cry2Ab targeted to the chloroplasts or a combination of Cry1A and Cry2Ab. All events exhibited significantly better fall armyworm control than the control plants (p≦0.05).

TABLE 7
Leaf Damage Rating of Transgenic Maize Expressing
Cry1Bb Infested with Fall Armyworm.

| | | FAW^{A} | |
|---|---|---|---|
| Gene | Event | 0-9 leaf | SE⁸ |
| 1Bb | RAB172 | 0.33 | 0.38 |
| 1Bb | RAB401 | 1.78 | 0.38 |
| 1Bb | RAB150 | 0.75 | 0.38 |
| 2Ab | MON840 | 0.03 | 0.38 |
| 1A/2Ab | MON810/840 | 0.00 | 0.38 |
| Control | B73/H99 | 3.33 | 0.38 |

A:leaf damage rating scale of 0-9 where 0 represents no damage/ excellent control and a 9 represents extreme damage no control.
B:SE indicates standard error or standard deviation from the indicated leaf damage rating

### Example 7

### Lepidopteran Pest Control by Plants Expressing Cry1Bb

Leaf disks from V4 stage transgenic maize plants were exposed to corn earworm (CEW), fall armyworm (FAW), black cutworm (BCW), and European corn borer (ECB) under controlled conditions to determine the effect of in planta expression of insecticidal amounts of a variant Cry1Bb insecticidal amino acid sequence. Expression levels of Cry1Bb protein was determined from disks derived from the same leaves used for the bioassay. Eight sibling plants per event were evaluated for insecticidal activity as measured using the leaf damage rating (LDR) scale of 0-11 (0 is complete control; 11 is no control, with intermediated levels defined as excellent, good, and marginal). Plants expressing Cry1Bb exhibited excellent control of ECB, good control of FAW, marginal control of CEW, and no control of BCW (Table 8). Some control of CEW was also observed with leaf disks from plants transformed with pMON33750, an unexpected result in view of previous diet incorporation assays where CEW was challenged with solubilized Cry1Bb derived from Bacillus thuringiensis. Leaf disks derived from the commercial event expressing Cry1A, MON810, were used as the positive control and displayed excellent control of both ECB and CEW, but no control of FAW, which highlights the utility of the Cry1Bb transgene in FAW control. Maize event MON840 expressing a gene encoding a chloroplast targeted Cry2Ab insecticidal crystal protein was a positive check for control of each of the target pests in this study.

TABLE 8
Bioactivity of Cry1Bb Transgenic Maize Against CEW, FAW, BCW, and ECB
R1 generation Cry1Bb transgenic plants leaf disk bioassay study.

| | | CEW | FAW | BCW | ECB | Expression |
|---|---|---|---|---|---|---|
| Plant | Event | LDR (0-11) | LDR (0-11) | LDR (0-11) | LDR (0-11) | “cry1Bb, ppm” |
| RR99MJV03:438:1 | RAB114 | 4 | 2 | 8 | 1 | 5.64 |
| RR99MJV03:438:2 | RAB114 | 4 | 1 | 5 | 0 | 4.43 |
| RR99MJV03:438:3 | RAB114 | 5 | 4 | 11 | 1 | 5.19 |
| RR99MJV03:438:4 | RAB114 | 7 | 1 | 7 | 0 | 6.73 |
| RR99MJV03:438:5 | RAB114 | 6 | 4 | 11 | 0 | 4.42 |
| RR99MJV03:438:6 | RAB114 | 6 | 3 | 11 | 0 | 3.05 |
| RR99MJV03:438:7 | RAB114 | 4 | 1 | 11 | 0 | 3.41 |
| RR99MJV03:438:8 | RAB114 | 8 | 5 | 11 | 1 | 1.19 |
| RR99MJV03:441:1 | RAB138 | 6 | 11 | 11 | 0 | 1.45 |
| RR99MJV03:441:2 | RAB138 | 4 | 1 | 11 | 0 | 1.61 |
| RR99MJV03:441:3 | RAB138 | 8 | 4 | 11 | 0 | 2.86 |
| RR99MJV03:441:4 | RAB138 | 11 | 2 | 11 | 0 | 2.75 |
| RR99MJV03:441:5 | RAB138 | 11 | 3 | 11 | 0 | 2.87 |
| RR99MJV03:441:6 | RAB138 | 4 | 1 | 11 | 0 | 1.48 |
| RR99MJV03:441:7 | RAB138 | 4 | 1 | 11 | 0 | 1.45 |
| RR99MJV03:441:8 | RAB138 | 11 | 4 | 11 | 1 | 1.59 |
| RR99MJV03:473:1 | RAB169 | 11 | 2 | 11 | 0 | 5.39 |
| RR99MJV03:473:2 | RAB169 | 6 | 1 | 9 | 0 | 4.96 |
| RR99MJV03:473:3 | RAB169 | 5 | 3 | 8 | 0 | 5.09 |
| RR99MJV03:473:4 | RAB169 | 7 | 3 | 8 | 1 | 3.62 |
| RR99MJV03:473:5 | RAB169 | 5 | 1 | 7 | 1 | 7.15 |
| RR99MJV03:473:6 | RAB169 | 11 | 1 | 11 | 0 | 3.89 |
| RR99MJV03:473:7 | RA8169 | 10 | 4 | 11 | 0 | 6.08 |
| RR99MJV03:473:8 | RAB169 | 3 | 1 | 8 | 1 | 12.74 |
| RR99MJV03:477:1 | RAB174 | 11 | 5 | 11 | 0 | 6.35 |
| RR99MJV03:477:2 | RAB174 | 11 | 3 | 11 | 0 | 4.19 |
| RR99MJV03:477:3 | RAB174 | 11 | 2 | 11 | 1 | 6.93 |
| RR99MJV03:477:4 | RAB174 | 7 | 4 | 11 | 0 | 5.57 |
| RR99MJV03:477:5 | RAB174 | 11 | 2 | 11 | 0 | 3.92 |
| RR99MJV03:477:6 | RAB174 | 8 | 1 | 11 | 0 | 6.31 |
| RR99MJV03:477:7 | RAB174 | 4 | 3 | 11 | 0 | 4.25 |
| RR99MJV03:477:8 | RAB174 | 10 | 1 | 11 | 0 | 3.66 |
| RR99MJV03:483:1 | RAB180 | 4 | 2 | 11 | 0 | 8.58 |
| RR99MJV03:483:2 | RAB180 | 2 | 2 | 7 | 0 | 6.94 |
| RR99MJV03:483:3 | RAB180 | 3 | 3 | 11 | 0 | 5.35 |
| RR99MJV03:483:4 | RAB180 | 11 | 5 | 11 | 0 | 5.02 |
| RR99MJV03:483:5 | RAB180 | 4 | 1 | 7 | 0 | 13.68 |
| RR99MJV03:483:6 | RAB180 | 11 | 2 | 8 | 0 | 9.67 |
| RR99MJV03:483:7 | RAB180 | 4 | 4 | 11 | 0 | 4.22 |
| RR99MJV03:483:8 | RAB180 | 4 | 0 | 11 | 0 | 3.81 |
| RR99MJV03:490:1 | RAB186 | 4 | 1 | 6 | 0 | 8.32 |
| RR99MJVQ3:490:2 | RAB186 | 11 | 1 | 11 | 0 | 8.59 |
| RR99MJV03:490:3 | RAB186 | 11 | 8 | 11 | 0 | 6.79 |
| RR99MJV03:490:4 | RAB186 | 11 | 0 | 11 | 0 | 4.8 |
| RR99MJV03:490:5 | RAB186 | 6 | 2 | 11 | 0 | 8.05 |
| RR99MJV03:490:6 | RAB186 | 8 | 4 | 6 | 0 | 13 |
| RR99MJV03:490:7 | RAB186 | 11 | 1 | 9 | 0 | 4.12 |
| RR99MJV03:490:8 | RAB186 | 5 | 0 | 10 | 0 | 3.51 |
| RR99MJV03:492:1 | RAB187 | 8 | 1 | 6 | 0 | 5.88 |
| RR99MJV03:492:2 | RAB187 | 10 | 1 | 9 | 0 | 9.26 |
| RR99MJV03:492:3 | RAB187 | 4 | 1 | 6 | 0 | 4.76 |
| RR99MJV03:492:4 | RAB187 | 3 | 1 | 8 | 0 | 3.84 |
| RR99MJV03:492:5 | RAB187 | 5 | 2 | 7 | 0 | 4.7 |
| RR99MJV03:492:6 | RAB187 | 8 | 1 | 8 | 0 | 4.42 |
| RR99MJV03:492:7 | RAB187 | 11 | 2 | 5 | 0 | 4.71 |
| RR99MJV03:492:8 | RAB187 | 3 | 9 | 6 | 0 | 3.28 |
| RR99MJV03:499:1 | RAB196 | 2 | 1 | 11 | 0 | 5.76 |
| RR99MJV03:499:2 | RAB196 | 7 | 2 | 11 | 0 | 6.73 |
| RR99MJV03:499:3 | RAB196 | 4 | 7 | 11 | 2 | 5.07 |
| RR99MJV03:499:4 | RAB196 | 8 | 3 | 11 | 0 | 5.13 |
| RR99MJV03:499:5 | RAB196 | 11 | 3 | 11 | 0 | 4.62 |
| RR99MJV03:499:6 | RAB196 | 3 | 1 | 11 | 0 | 5.11 |
| RR99MJV03:499:7 | RAB196 | 8 | 2 | 11 | 1 | 4.38 |
| RR99MJV03:499:8 | RAB196 | 9 | 1 | 11 | 0 | 3.09 |
| RR99MJV03:500:1 | RAB196 | 11 | 11 | 2 | 0 | 4.25 |
| RR99MJV03:500:3 | RAB196 | 7 | 1 | 11 | 0 | 4.86 |
| RR99MJV03:500:4 | RAB196 | 6 | 2 | 5 | 0 | 2.95 |
| LH198 (row9) | — | 11 | 11 | 6 | 11 | neg. control |
| A1 (row 10) | — | 11 | 9 | 11 | 11 | neg. control |
| Control | Mon810 | 1 | 11 | 11 | 1 | cry1Ab |
| Control | Mon840 | 0 | 0 | 0 | 0 | cry2Ab |

### Example 8

### Cry1Bb Transgenic Plants Display Improved Insect Resistance Management Characteristics under Laboratory and Field Conditions

A plant transformation vector containing a Cry1Bb coding sequence as set forth in SEQ ID NO:3 operably linked upstream to a CaMV35S promoter (P-e35S) and a wheat chlorophyll ab binding protein untranslated leader sequence (L-TaCAB) and downstream to a nopaline synthase 3′ end transcription termination and polyadenylation sequence (T-AGRtu.nos) was used to produce Brassica sp. transformation events expressing Cry1Bb amino acid sequence variant insecticidal protein. These plants were assayed for the ability to control Cry1A-type resistant Diamondback moth (DBM) infestation. Transgenic Brassica sp (Broccoli and Cauliflower) was obtained by Agrobacterium mediated transformation of cotyledonary petioles and selection on media containing kanamycin. Transgenic events expressing Cry1Bb were identified by ELISA analysis. Brassica sp. transgenic events were also produced by Agrobacterium mediated transformation methods using a kanamycin selectable plant transformation vector which contained an expression cassette comprising a synthetic sequence encoding a Cry1Ac insecticidal protein operably linked upstream to a CamV35S promoter sequence (P-CaMV35S) and a petunia species Hsp70 untranslated leader sequence (L-Pet.Hsp70) and a 3′ end plant functional transcription termination and polyadenylation sequence.

Cry1Bb transgenic Brassica sp. plants were challenged in controlled laboratory conditions where insect mortality could be accurately monitored. Broccoli plants expressing Cry1Ac were used as controls and were infested in parallel with the transgenic plants expressing Cry1Bb. Plants were challenged with cabbage looper, diamondback moth (DBM), Cry1C-resistant diamondback moth (1CrDBM), and Cry1A resistant diamondback moth (both plant varieties displayed excellent insecticidal bioactivity against cabbage looper, diamondback moth (DBM), and Cry1C-resistant diamondback moth (1ArDBM) (Table 9). Three replicates were used per treatment, and there were twenty (20) larvae per replicate to each plant event. Infestation temperature was maintained at 27 C throughout each treatment, and the results were determined at seventy-two (72) hours after infestation. Only the plants expressing Cry1Bb exhibited insecticidal activity against the 1ArDBM. Transgenic cauliflower expressing Cry1Bb also displayed excellent control of all species tested. Cabbage Looper was also controlled in Cry1Bb cauliflower events #2 and #3.

TABLE 9
Insecticidal Bioactivity of Transgenic Brassica Plants
Expressing Cry 1Ac or Cry1Bb

| | | | | Cabbage |
|---|---|---|---|---|
| Event | DBM | 1ArDBM | 1CrDBM | Looper |
| | % mortality (SEM) | | | |
| Broccoli | | | | |
| Cry1Ac #1 | 100 (0)^{a} | 5.00 (2.87)^{b} | 100 (0)^{a} | 100 (0)^{a} |
| Cry1Ac #2 | 100 (0)^{a} | 6.67 (1.67)^{b} | 96.7 (1.67)^{a} | 100 (0)^{a} |
| Cry1Bb #1 | 100 (0)^{a} | 100 (0)^{a} | 100 (0)^{a} | 100 (0)^{a} |
| Cry1Bb #2 | 100 (0)^{a} | 100 (0)^{a} | 100 (0)^{a} | 100 (0)^{a} |
| Cry1Bb #3 | 100 (0)^{a} | 100 (0)^{a} | 100 (0)^{a} | 61.7 (21)^{b} |
| Cry1Bb #4 | 100 (0)^{a} | 100 (0)^{a} | 100 (0)^{a} | 80 (2.9)^{ab} |
| non-transgenic | 0 (0)^{a} | 3.33 (1.67)^{b} | 5.0 (0)^{b} | 3.3 (1.67)^{c} |
| control | | | | |
| Cauliflower | | | | |
| Cry1Bb #1 | 100 (0)^{a} | 88 (12)^{b} | 72 (15)^{c} | 15 (5.0)^{efg} |
| Cry1Bb #2 | 100 (0)^{a} | 100 (0)^{a} | 92 (8.3)^{ab} | 93 (6.7)^{ab} |
| Cry1Bb #3 | 100 (0)^{a} | 100 (0)^{a} | 97 (3.3)^{a} | 100 (0)^{a} |
| Cry1Bb#4 | 100 (0)^{a} | 92 (8.3)^{ab} | 73 (16)^{bc} | 47 (21)^{cde} |
| Cry1Bb #5 | 100 (0)^{a} | 100 (0)^{a} | 93 (3.3)^{a} | 43 (28)^{cde} |
| non-transgenic | 1.7 (1.7)^{b} | 0 (0)^{c} | 3.3 (3.3)^{d} | 1.67 (1.67)^{g} |
| control | | | | |

values in a column followed by the same superscript letter are not significantly different from the other values in the column (P < 0.05, LSD); Numbers in parenthesis indicate the extent of variation of the results in that particular replicate.

Transgenic Brassica sp. were also tested under field conditions for resistance to endemic Lepidopteran insect pest infestations. Typical insect infestations in the test location near Weslaco, Texas were initiated in the fall season and included cabbage looper, DBM, beet armyworm, and the great southern white butterfly. Plants were seeded in September and evaluated in December. Plants were evaluated for the numbers of insect larvae per plant and for the extent of insect feeding damage. Damage was assessed on ten plants per transgenic event based on the following zero (0) to five (5) scale: 0—no damage, 1—minor feeding damage (1% consumed by infesting larvae), 2—minor to moderate damage (2-5% consumed by infesting larvae), 3—moderate damage (6-10% consumed by infesting larvae), 4—moderate to heavy damage (11-30% consumed by infesting larvae) and 5—heavy damage (>30% consumed by infesting larvae). The results are shown in Table 10. The data demonstrate that both transgenic broccoli and cauliflower transformed to express Cry1Bb or amino acid sequence variants exhibit statistically significant reductions in the number of lepidopteran pest larvae per plant and in the level of insect damage endured over the course of the growing season. In broccoli, field performance of plants expressing the transgene encoding a Cry1Bb protein was indistinguishable from field performance of plants expressing the transgene encoding a Cry1Ac protein.

TABLE 10
Field Tests of Lepidopteran Insect Pest Infestation on Transgenic Brassica
Plants Expressing Cry1Ac or Cry1Bb

| | mean larvae/plant (N) | Mean Damage (N) |
|---|---|---|
| Broccoli | | |
| Cry1Ac #1 | NT | NT |
| Cry1Ac #2 | 0.63 (19)^{a} | 0.46 (3)^{a} |
| Cry1Bb #1 | 0.21 (24)^{a} | 0.48 (3)^{a} |
| Cry1Bb #2 | NT | NT |
| Cry1Bb #3 | NT | NT |
| Cry1Bb #4 | 0.13 (15)^{a} | 0.43 (3)^{a} |
| 987146-004 | 14 (19)^{b} | 1.9 (3)^{b} |
| (neg. Ctrl.) | | |
| non-transgenic | 1.3 (43)^{b} | 1.9 (5)^{b} |
| Cauliflower | | |
| Cry1Bb #1 | 0.12 (25)^{a} | 0.0 (3)^{a} |
| Cry1Bb #2 | 0.21 (19)^{b} | 0.07 (3)^{a} |
| Cry1Bb #3 | 0.00 (4)^{a} | 1.25 (1)^{c} |
| Cry1Bb #4 | 0.31 (26)^{b} | 0.52 (3)^{c} |
| Cry1Bb #5 | 0.29 (17)^{b} | 0.30 (3)^{b} |
| non-transgenic | 1.6 (41)^{c} | 2.1 (5)^{d} |

values in a column followed by the same superscript letter are not significantly different from the other values in the column (P<0.05, LSD); Numbers in parenthesis indicate the extent of variation of the results in that particular replicate.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications and patents mentioned in this specification are herein incorporated by reference as if each individual publication or patent was specially and individually stated herein to be incorporated by reference.

1. A polynucleotide sequence optimized for expression of an insecticidal protein in a plant wherein said polynucleotide sequence comprises a sequence selected from the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13.

2. The polynucleotide sequence according to claim 1 wherein said sequence is SEQ ID NO:3 from about nucleotide position 7 through about nucleotide position 1803.

3. The polynucleotide sequence according to claim 1 wherein said sequence is SEQ ID NO:5 from about nucleotide position 2650 through about nucleotide position 4446.

4. The polynucleotide sequence according to claim 1 wherein said sequence is SEQ ID NO:8 from about nucleotide position 3047 through about nucleotide position 4844.

5. The polynucleotide sequence according to claim 1 wherein said sequence is SEQ ID NO:11 from about nucleotide position 1247 through about nucleotide position 3043.

6. The polynucleotide sequence according to claim 1 wherein said sequence is SEQ ID NO:13 from about nucleotide position 1658 through about nucleotide position 3454.

7. A polynucleotide sequence encoding an insecticidal protein, said protein being selected from the group consisting of SEQ ID NO:2 from about amino acid position 2 through about amino acid position 600, SEQ ID NO:4 from about amino acid position 3 through about amino acid position 601, SEQ ID NO:7 from about amino acid position 3 through about amino acid position 601, SEQ ID NO:10 from about amino acid position 3 through about amino acid position 601, SEQ ID NO:12 from about amino acid position 3 through about amino acid position 601, and SEQ ID NO:14 from about amino acid position 3 through about amino acid position 601.

8. The polynucleotide sequence of claim 7 wherein said polynucleotide sequence encoding said protein is selected from the group consisting of SEQ ID NO:3, SEQ ID NO:5, SEQ ID NO:8, SEQ ID NO:11, and SEQ ID NO:13.

9. A expression cassette comprising the polynucleotide sequence substantially as set forth in SEQ ID NO:3 which functions in plants to produce an insecticidal protein, wherein said expression cassette is selected from the group consisting of SEQ ID NO:5, SEQ ID NO:8, SEQ ID NO:11, and SEQ ID NO:13.

10. A plant comprising a polynucleotide sequence optimized for expression of an insecticidal protein in a plant wherein said polynucleotide sequence comprises a sequence selected from the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13.

11. A seed or progeny produced from the plant of claim 10, wherein said seed or progeny comprises said sequence selected from the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13.

12. A plant cell comprising a polynucleotide sequence optimized for expression of an insecticidal protein in a plant wherein said polynucleotide sequence comprises a sequence selected from the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13.

13. A method for producing a transgenic plant cell expressing an insecticidal Cry1Bb endotoxins fragment, said method comprising transforming a plant cell with a polynucleotide sequence comprising a plant functional promoter operably linked to a nucleotide sequence encoding said fragment wherein said nucleotide sequence is selected from the group consisting of from about nucleotide position 7 through about nucleotide position 1803 as set forth in SEQ ID NO:3, from about nucleotide position 2650 through about nucleotide position 4446 as set forth in SEQ ID NO:5, from about nucleotide position 3047 through about nucleotide position 4844 as set forth in SEQ ID NO:8, from about nucleotide position 1247 through about nucleotide position 3043 as set forth in SEQ ID NO:11, and from about nucleotide position 1658 through about nucleotide position 3454 as set forth in SEQ ID NO:13.

14. A method for producing a transgenic plant resistant to lepidopteran insect infestation comprising:
a) transforming a plant cell with a polynucleotide sequence comprising a plant functional promoter operably linked to a nucleotide sequence encoding an insecticidal. Cry1Bb delta endotoxin fragment; and
b) regenerating a transgenic plant from said plant cell, wherein said transgenic plant comprises said polynucleotide sequence and expresses insecticidally effective amounts of said fragment.

15. A method for producing a transgenic plant resistant to insect infestation comprising breeding together
a) a first plant transformed to contain a first nucleotide sequence encoding a first Bt insecticidal protein and a first selectable marker with
b) a second plant transformed to contain a second nucleotide sequence different from the first, wherein said second nucleotide sequence encodes a second Bt insecticidal protein different from the first, and a second selectable marker different from the first
wherein said transgenic plant comprises both the first and the second nucleotide sequences;
wherein the first and the second selectable markers are selected from the group consisting of antibiotic resistance genes, herbicide resistance genes, and genes encoding enzymes that react with a substrate to form a product that is visually or immunologically observable;
wherein the first Bt insecticidal protein comprises an insecticidal fragment of a Cry1Bb protein as set forth in SEQ ID NO:3 from about nucleotide position 7 through about nucleotide position 1803; and
wherein the second Bt insecticidal protein is selected from the group of toxins consisting of a Cry1, Cry2, Cry3, Cry4, Cry5, Cry6, Cry9, Cry22, a Cry binary toxin, a VIP toxin, a TIC901 or related toxin, and combinations thereof.

16. The method of claim 15 wherein said herbicide resistance genes are selected from the group consisting of a gox gene, a gene encoding an EPSPS that is insensitive to glyphosate inhibition, a phnO gene, a bar gene, and a glyphosate acetylase gene.

17. A nucleotide sequence encoding at least an insecticidal fragment of a Cry1Bb delta endotoxin protein, said protein comprising an amino acid sequence as set forth in SEQ ID NO:4 from about amino acid position 3 through about amino acid position 601, wherein said nucleotide sequence hybridizes under stringent conditions with a nucleotide sequence as set forth in SEQ ID NO:3 from about nucleotide position 7 through about nucleotide position 1803.

18. A composition comprising an insecticidally effective amount of a Cry1Bb endotoxin protein or insecticidal fragment thereof expressed in a plant from a segment of a nucleotide sequence as set forth in SEQ ID NO:3 from about nucleotide position 7 through about nucleotide position 1803 or from a nucleotide sequence encoding said protein or fragment thereof that hybridizes to said segment.

19. A biological sample derived from a plant, tissue, or seed, wherein said sample comprises a nucleotide sequence which is or is complementary to a sequence selected from the group consisting of SEQ ID NO:3, SEQ ID NO:5, SEQ ID NO:8, SEQ ID NO:11, and SEQ ID NO:13, and wherein said sequence is detectable in said sample using a nucleic acid amplification or nucleic acid hybridization method.

20. The biological sample of claim 19 wherein said sample is selected from the group consisting of corn flour, corn meal, corn syrup, corn oil, corn starch, and cereals manufactured in whole or in part to contain corn by-products.

21. An extract derived from a corn plant, tissue, or seed comprising a nucleotide sequence which is or is complementary to a nucleotide sequence selected from the group consisting of SEQ ID NO:3, SEQ ID NO:5, SEQ ID NO:8, SEQ ID NO:11, and SEQ ID NO:13.

22. The extract of claim 21 wherein said sequence is detectable in said extract using a nucleic acid amplification or nucleic acid hybridization method.
